(19) United States
(12) Patent Application Publication
(10) Pub. No.: US 2023/0018737 A1
(43) Pub. Date: Jan. 19, 2023

(54) AN IN VITRO KIT AND A METHOD FOR DIAGNOSTICS IN TRANSPLANTS AND/OR AUTOIMMUNE DISEASES AND/OR TUMOR DISEASES AND/OR VACCINATIONS AND A MESSENGER-SUBSTANCE-CONTAINING SUPERNATANT, CELL-CONTAINING SEDIMENT, AND A COMBINATION, FOR USE IN DIAGNOSTICS IN TRANSPLANTS AND/OR AUTOIMMUNE DISEASES AND/OR TUMOR DISEASES AND/OR VACCINATIONS

(71) Applicant: HOT Screen GmbH, Reutlingen (DE)

(72) Inventor: Manfred Schmolz, Jettenburg (DE)

(21) Appl. No.: 17/761,489

(22) PCT Filed: Sep. 16, 2020
(86) PCT No.: PCT/EP2020/075894

§ 371 Date: Mar. 17, 2022

(30) Foreign Application Priority Data
Sep. 17, 2019 (DE) 10 2019 214 137.4

(51) Int. Cl. G01N 33/50; G01N 33/569; G01N 33/564

(52) U.S. Cl. CPC G01N 33/5047; G01N 33/56972; G01N 33/564; G01N 2800/52

(57) ABSTRACT

The invention relates to the use in vitro of a kit
for diagnostics, more particularly therapy-accompanying diagnostics, relating to a transplant
and/or
for diagnostics, more particularly therapy-accompanying diagnostics, relating to an autoimmune disease
and/or
for diagnostics, more particularly therapy-accompanying diagnostics, relating to a tumor disease
and/or
for diagnostics, more particularly therapy-accompanying diagnostics, relating to a vaccination,
wherein the kit has the following components:
a blood collection container, a blood collection set or blood collection system including a blood collection container,
a nutrient medium for blood, and
at least one activator for activating blood cells, more particularly immune cells, and/or at least one inhibitor for inhibiting blood cells, more particularly immune cells, and/or at least one modulator for modulating blood cells, more particularly immune cells.

The invention further relates to the use in vitro of a method for diagnostics in transplants and/or autoimmune diseases and/or tumor diseases and/or vaccinations, to a messenger-substance-containing supernatant for use in vitro in diagnostics in transplants and/or autoimmune diseases and/or tumor diseases and/or vaccinations, and to a combination for use in diagnostics in transplants and/or autoimmune diseases and/or tumor diseases and/or vaccinations.

### TECHNICAL FIELD

This disclosure relates to the use in vitro of a kit for diagnostics in transplants and/or autoimmune diseases and/or tumor diseases and/or vaccinations, to the use in vitro of a method for diagnostics in transplants and/or autoimmune diseases and/or tumor diseases and/or vaccinations, to a messenger-substance-containing supernatant or a cell-containing sediment for use in vitro in diagnostics in transplants and/or autoimmune diseases and/or tumor diseases and/or vaccinations, and to a combination for use in vitro in diagnostics in transplants and/or autoimmune diseases and/or tumor diseases and/or vaccinations.

### BACKGROUND

The generation of a blood count to determine the immune defense activity of blood and also the analysis of individual blood components have become important tools in in-vitro diagnostics, and in therapy-accompanying diagnostics in particular.

For the determination of immune defense activity, immune cells present in blood are usually investigated separately from the other blood components. This is sufficient for morphological/histological/histochemical analyses. However, if functional tests are to be carried out, which always have much higher informative value, it is important to investigate the immune cells by selectively executed cultures. The basis of physiological and pathophysiological responses of immune system cells in vivo is a highly complex interconnecting system of feedback mechanisms that adapts in a variable manner to demands in the individual example. The interconnecting system of feedback mechanisms includes messenger substances and their receptors, ion channels, signal transduction cascades docking to the receptors and ion channels, regulatory factors of gene expression, and enzymes that influence signal transduction, transcription, translation, post-translational modification, and protein release.

The problem with the analysis methods to date is that the aforementioned feedback mechanisms cannot adequately be taken into account. This in turn entails a risk of unreliable or even distorted analysis results.

A further disadvantage is that functional investigations of immune cells usually require time-consuming work steps, storage, and transport. This in turn increases the risk of impairment of the activity of the immune cells under investigation and thus of the occurrence of distorted analysis results. In extreme instances, this can make in-vitro therapy-accompanying diagnostics unreliable.

It could therefore be helpful to provide means for diagnostics in transplants and/or autoimmune diseases and/or tumor diseases and/or vaccinations that largely avoid known disadvantages.

The wording of all the appended claims is hereby incorporated in the content of this disclosure by express reference.

According to a first aspect, this disclosure relates to the use in vitro of an in vitro kit

for diagnostics, more particularly therapy-accompanying diagnostics, relating to a transplant, more particularly for the prognostic assessment of the prospects of success and/or predicting a clinical outcome of a transplant and/or determining the suitability and/or effectiveness and/or dose of compounds, more particularly immunosuppressants, for a transplant, and/or therapy monitoring, i.e., assessing and/or monitoring the course of a transplant,

and/or

for diagnostics, more particularly therapy-accompanying diagnostics relating to an autoimmune disease, more particularly characterizing the stage of an autoimmune disease and/or the prognostic assessment of the prospects of success and/or for predicting a clinical outcome of a therapy, i.e., of a prophylaxis and/or of a treatment of an autoimmune disease and/or determining the suitability and/or effectiveness and/or dose of compounds, more particularly immunosuppressants and/or anti-inflammatories, for a therapy, i.e., prophylaxis and/or treatment, of an autoimmune disease and/or therapy monitoring, i.e., assessing and/or monitoring the course of an autoimmune disease,

and/or

for diagnostics, more particularly therapy-accompanying diagnostics, relating to a tumor disease, more particularly for the prognostic assessment of the prospects of success and/or predicting a clinical outcome of a therapy, i.e., of a prophylaxis and/or of a treatment of a tumor disease and/or determining the suitability and/or effectiveness and/or dose of compounds for a therapy, i.e., prophylaxis and/or treatment, of a tumor disease and/or therapy monitoring, i.e., assessing and/or monitoring the course of a tumor disease,

and/or

for diagnostics, more particularly therapy-accompanying diagnostics, relating to a vaccination (immunization), more particularly for the prognostic assessment of the prospects of success and/or predicting a clinical outcome of a vaccination (immunization) and/or therapy monitoring, i.e., assessing and/or monitoring the course of a vaccination (immunization), wherein the kit has the following components:

a blood collection container, more particularly a syringe, or a blood collection set or blood collection system including a blood collection container, more particularly a syringe,

a nutrient medium for blood, more particularly whole blood, and

at least one activator that activates or stimulates blood cells, more particularly immune cells, and/or at least one inhibitor that inhibits or suppresses blood cells, more particularly immune cells, and/or at least one modulator that modulates or influences blood cells, more particularly immune cells.

We surprisingly found that a kit is suitable for therapy-accompanying diagnostics in transplants, autoimmune diseases, tumor diseases, and vaccinations. A particular advantage here is that the kit can be used to check the activity of immune cells in their natural environment, i.e., in whole blood. This permits execution-related artefacts, as can occur with generic methods of analysis, to be almost completely avoided. In addition, it is possible to maximize standardization of the culture conditions and thus the analysis results obtained with the method. A further advantage is that the kit allows culture of immune cells under conditions close to those in vivo (“organ-typical culture”), which results in additional improvement of the analysis results obtained. A further advantage is that the kit permits investigation of cellular activities under activating, i.e., mostly inflammatory and thus pathophysiologically relevant, conditions. Another advantage of the kit relates to the ability to selectively adapt activation conditions to a wide range of challenges associated with the abovementioned in-vitro diagnostics.

The expression “whole blood” should mean blood, more particularly a blood sample, wherein the blood, and more particularly the blood sample, contains all blood components physiologically occurring in blood, more particularly in human blood, more particularly cells (so-called blood cells), preferably immune cells.

The expression “blood cells” means cells occurring in blood, more particularly whole blood, which may be selected more particularly from the group consisting of monocytes, macrophages, mast cells, antigen-presenting cells, neutrophils, eosinophils, basophils, B lymphocytes, plasma cells, memory B cells, T helper cells, cytotoxic T cells, regulatory T helper cells, memory T cells, killer T cells, natural killer (NK) cells, platelets, and mixtures of at least two of the blood cells.

The expression “immune cells” means more particularly cells selected from the group consisting of monocytes, macrophages, mast cells, antigen-presenting cells, neutrophils, eosinophils, basophils, B lymphocytes, plasma cells, memory B cells.

T helper cells, cytotoxic T cells, regulatory T helper cells, memory T cells, killer T cells, natural killer (NK) cells, and mixtures of at least two of the immune cells.

The expression “activator for activating or stimulating blood cells, more particularly immune cells” means more particularly a compound or cell capable of the activation or stimulation of cells occurring in the blood, more particularly immune cells, more particularly of the activation or stimulation, i.e., of the triggering/provoking and/or enhancement, through or in cells occurring in the blood (so-called blood cells), more particularly immune cells, of a production or secretion of cytokines, an expression of mRNA (messenger RNA), an expression of surface markers (cell surface molecules), a change in ion currents across the cell membrane, an expression of intracellular activation markers, a change in the phosphorylation pattern of components of signal transduction cascades or a translocation of gene-regulating elements. The activator may also be referred to as a blood cell activator, more particularly an immune cell activator, or a blood cell stimulant, more particularly an immune cell stimulant.

The expression “at least one activator for activating or stimulating blood cells, more particularly immune cells” may mean just one activator type or a combination, more particularly mixture, of different activator types.

The expression “inhibitor for inhibiting or suppressing blood cells, more particularly immune cells” means more particularly a compound or cell capable of the inhibition or suppression of cells occurring in the blood (so-called blood cells), more particularly immune cells, more particularly of the inhibition or suppression, i.e., of the prevention/subduing or reduction, through cells occurring in the blood, more particularly immune cells, of a production or secretion of cytokines, an expression of mRNA (messenger RNA), an expression of surface markers (cell surface molecules), a change in ion currents across the cell membrane, an expression of intracellular activation markers, a change in the phosphorylation pattern of components of signal transduction cascades or a translocation of gene-regulating elements. The inhibitor may also be referred to as a blood cell inhibitor, more particularly an immune cell inhibitor, or a blood cell suppressor, more particularly an immune cell suppressor, or as an anti-inflammatory.

The expression “at least one inhibitor for inhibiting or suppressing blood cells, more particularly immune cells” may mean just one inhibitor type or a combination, more particularly mixture, of different inhibitor types.

The expression “modulator for modulating or influencing blood cells, more particularly immune cells” means more particularly a compound or cell capable of the concentration- and/or environment-dependent activation or inhibition of cells occurring in the blood (so-called blood cells), more particularly immune cells, more particularly of the activation/stimulation or inhibition/suppression, through cells occurring in the blood, more particularly immune cells, of a production or secretion of cytokines, an expression of mRNA (messenger RNA), an expression of surface markers (cell surface molecules), a change in ion currents across the cell membrane, an expression of intracellular activation markers, a change in the phosphorylation pattern of components of signal transduction cascades or a translocation of gene-regulating elements.

The expression “at least one modulator for modulating or influencing blood cells, more particularly immune cells” may mean just one modulator type or a combination, more particularly mixture, of different modulator types.

The expression “messenger substance” means a compound that serves for signal transduction or chemical communication (chemocommunication). The messenger substance may be a low-molecular-weight compound such as histamine, serotonin, kynurenine, prostaglandins, leukotrienes, ATP, ADP, AMP, oxygen radicals, and the like, and/or a high-molecular-weight compound such as a cytokine, chemokine, growth factor or the like. The messenger substance may also be referred to as a mediator.

The expression “nutrient medium” means a culture medium suitable for culturing blood, more particularly whole blood.

The nutrient medium is preferably present in liquid form. Alternatively, the nutrient medium may be present in a solid, especially gelled, form. In addition to water, the nutrient medium may by preference comprise nutrients and trace elements, a dye such as phenol red, an indicator such as a pH indicator, and a buffer substance or a mixture of different buffer substances.

The expression “diagnostics relating to a transplant” means any type of test, more particularly functional test, or investigation, more particularly functional investigation, in vitro in the context of a transplant.

The expression “diagnostics relating to an autoimmune disease” means any type of test, more particularly functional test, or investigation, more particularly functional investigation, in vitro in the context of an autoimmune disease.

The expression “diagnostics relating to a tumor disease” means any type of test, more particularly functional test, or investigation, more particularly functional investigation, in vitro in the context of a tumor disease.

The expression “diagnostics relating to a vaccination” means any type of test, more particularly functional test, or investigation, more particularly functional investigation, in vitro in the context of a vaccination.

The expression “functional test” means a test that involves the observation or testing of blood cells, more particularly immune cells, in different situations, more particularly by incubating with at least one activator that activates or stimulates blood cells, more particularly immune cells, and/or at least one inhibitor that inhibits or suppresses blood cells, more particularly immune cells, and/or a modulator that modulates or influences blood cells, more particularly immune cells.

The expression “functional investigation” means an investigation that involves the observation or investigation of blood cells, more particularly immune cells, in different situations, more particularly by incubating with at least one activator that activates or stimulates blood cells, more particularly immune cells, and/or at least one inhibitor that inhibits or suppresses blood cells, more particularly immune cells, and/or at least one modulator that modulates or influences blood cells, more particularly immune cells.

The expression “therapy-accompanying diagnostics relating to a transplant” means more particularly the in-vitro prognostic assessment of the prospects of success and/or the in-vitro prediction of a clinical outcome of a transplant and/or the in-vitro determination of the suitability and/or effectiveness and/or dose of compounds, more particularly immune-suppressants, for a transplant and/or the in-vitro therapy monitoring of a transplant, i.e., the assessment and/or monitoring of the course of a transplant and/or of an accompanying therapy.

The expression “therapy-accompanying diagnostics relating to an autoimmune disease” means more particularly the in-vitro characterization of the stage of an autoimmune disease and/or the in-vitro prognostic assessment of the prospects of success and/or the in-vitro prediction of a clinical outcome of a therapy, i.e., of a prophylaxis and/or of a treatment of an autoimmune disease and/or the in-vitro determination of the suitability and/or effectiveness and/or dose of compounds, more particularly immunosuppressants, for a therapy, i.e., for prophylaxis and/or treatment, of an autoimmune disease and/or the in-vitro therapy monitoring of an autoimmune disease, i.e., the assessment and/or monitoring of the course of a therapy of an autoimmune disease.

The expression “therapy-accompanying diagnostics relating to a tumor disease” means more particularly the in-vitro prognostic assessment of the prospects of success and/or the in-vitro prediction of a clinical outcome of a therapy, i.e., of a prophylaxis and/or of a treatment of a tumor disease and/or the in-vitro determination of the suitability and/or effectiveness and/or dose of compounds, more particularly immunosuppressants, for a therapy, i.e., for a treatment, of a tumor disease and/or the in-vitro therapy monitoring of a tumor disease, i.e., the assessment and/or monitoring of the course of a therapy of a tumor disease.

The expression “therapy-accompanying diagnostics relating to a vaccination” means the in-vitro prognostic assessment of the prospects of success and/or the in-vitro prediction of a clinical outcome of a vaccination (immunization) and/or the in-vitro therapy monitoring, i.e., the assessment and/or monitoring of the course of a vaccination (immunization).

The at least one activator that activates or stimulates blood cells, more particularly immune cells, may be selected from the group consisting of cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the kit is used in vitro for diagnostics, more particularly therapy-accompanying diagnostics, relating to a transplant, more particularly for the prognostic assessment of the prospects of success and/or predicting a clinical outcome of a transplant and/or determining the suitability and/or effectiveness and/or dose of compounds, more particularly immunosuppressants, for a transplant, and/or therapy monitoring of a transplant.

The expression “transplant donor” may mean a human transplant donor or a nonhuman mammal.

The cells of the transplant donor are preferably organ cells, more particularly selected from the group consisting of blood cells, immune cells, skin cells, kidney cells, liver cells, heart cells, lung cells, pancreas cells, small intestine cells, bone cells, bone marrow cells, and combinations, more particularly mixtures, of at least two of the cells from a transplant donor. The cells of the transplant donor are particularly preferably blood cells, more particularly immune cells. With regard to suitable blood cells, more particularly immune cells, reference is made to the prior description.

The antigen preparations of cells from a transplant donor may be selected from the group consisting of intact cells, cell membrane fragments, microsomes, cell- and/or cell-fragment-free filtrates, ultrafiltrates, extracts, and combinations, more particularly mixtures, of at least two of the antigen preparations of cells from a transplant donor.

The antigens may be selected, for example, from the group consisting of cell membranes, components of cell membranes, proteins, glycoproteins, lipids, glycolipids, lipopeptides, and combinations, more particularly mixtures, of at least two of the antigens.

The expression “superantigens” means antigens that mediate direct binding of T-cell receptor (CD4-positive T lymphocyte) and MHC II molecules (antigen-presenting cells) without processing.

The superantigens may be selected, for example, from the group consisting of staphylococcal enterotoxins such as staphylococcal enterotoxin A and/or staphylococcal enterotoxin B and/or staphylococcal enterotoxin C, toxic shock syndrome toxin, Streptococcus pyogenes exotoxin, and combinations, more particularly mixtures, of at least two of the superantigens.

The mitogens may be selected, for example, from the group consisting of phytohemagglutinin, pokeweed mitogen, concanavalin A, and combinations, more particularly mixtures, of at least two of the mitogens.

The ligands (such as natural and/or semisynthetic and/or synthetic ligands) of blood cell surface receptors, more particularly immune cell surface receptors, that have an activating or inhibitory effect on blood cells, more particularly immune cells, may more particularly be ligands of CD (cluster of differentiation) molecules.

For example, the ligands (such as natural and/or semisynthetic and/or synthetic ligands) of blood cell surface receptors, more particularly immune cell surface receptors, that have an activating or inhibitory effect on blood cells, more particularly immune cells, may be selected from the group consisting of ligands of CD2, ligands of CD3, ligands of CD11, ligands of CD16, ligands of CD18, ligands of CD32, ligands of CD64, and combinations, more particularly mixtures, of at least two of the ligands. In particular, the ligands may be natural ligands of the aforementioned CD molecules such as LFA-3 and/or ICAM-1 and/or C3b and/or Fcγ.

The antibodies (such as agonistic and/or antagonistic antibodies) to blood cell surface receptors, more particularly immune cell surface receptors, that have an activating or inhibitory effect on blood cells, more particularly immune cells, may be selected, for example, from the group consisting of antibodies to CD2, antibodies to CD3, antibodies to CD11, antibodies to CD16, antibodies to CD18, antibodies to CD32, antibodies to CD64, and combinations, more particularly mixtures, of at least two of the antibodies.

The ligands (such as natural and/or semisynthetic and/or synthetic ligands) of intracellular receptors of blood cells, more particularly immune cells, that have an activating or inhibitory effect on blood cells, more particularly immune cells, may be selected, for example, from the group consisting of TLR ligands, NOD ligands, RIG ligands, PPAR-γ ligands, and combinations, more particularly mixtures, of at least two of the ligands.

The TLR ligands may be selected, for example, from the group consisting of lipopolysaccharides, endotoxins, R848, gardiquimod, compound A, compound B, VTX, poly-I:C, MDP, iE-DAP, CpG oligonucleotides, lipoproteins, lipoteichoic acids, flagellin, zymosan, derivatives of the TLR ligands, and combinations, more particularly mixtures, of at least two of the TLR ligands. In particular, the TLR ligands may be selected from the group consisting of lipopolysaccharides, endotoxins, R848, gardiquimod, poly-I:C, MDP, iE-DAP, CpG oligonucleotides, lipoproteins, lipoteichoic acids, flagellin, zymosan, derivatives of the TLR ligands, and combinations, more particularly mixtures, of at least two of the TLR ligands.

The NOD ligands may be selected, for example, from the group consisting of proteoglycan, MDP, iE-DAP, M-Tri-DAP, derivatives of the NOD ligands, and combinations, more particularly mixtures, of at least two of the NOD ligands.

The RIG ligands may be selected from the group consisting of viral dsRNA, analogs thereof such as poly-I:C, and preparations thereof.

In particular, the NOD ligands and/or RIG ligands may be single-stranded or double-stranded RNA.

The antibodies (such as agonistic and/or antagonistic antibodies) to intracellular receptors of blood cells, more particularly immune cells, that have an activating or inhibitory effect on blood cells, more particularly immune cells, may be selected, for example, from the group consisting of antibodies to TLR9, antibodies to NOD, antibodies to RIG, antibodies to PPAR-γ, and combinations, more particularly mixtures, of at least two of the antibodies.

The expression “adjuvants” means substances or mixtures of substances that serve to enhance antigen-specific immune system responses.

The adjuvants may be selected, for example, from the group consisting of poly-I:C, R848, CpG (especially in different variants), monophosphoryl lipid A, flagellin, Freund's adjuvant (complete or incomplete), QuilA, and combinations, more particularly mixtures, of at least two of the adjuvants.

The at least one inhibitor that inhibits or suppresses blood cells, more particularly immune cells, may be selected from the group consisting of anti-inflammatory compounds/drugs, immunosuppressants, and combinations, more particularly mixtures, of at least two of the inhibitors, when the kit is used in vitro for diagnostics, more particularly therapy-accompanying diagnostics, relating to a transplant, more particularly for the prognostic assessment of the prospects of success and/or for predicting a clinical outcome of a transplant and/or determining the suitability and/or effectiveness and/or dose of compounds, more particularly immunosuppressants, for a transplant, and/or for therapy monitoring of a transplant.

Anti-inflammatory compounds/drugs may be selected, for example, from the group consisting of cortisone, inhibitors of the synthesis of arachidonic acid metabolites, kinase inhibitors, inhibitors of cyclooxygenases, inhibitors of lipoxygenases, receptor blockers, siRNA, ion-channel blockers, and combinations, more particularly mixtures, of at least two of the anti-inflammatory compounds/drugs.

Immunosuppressants may be selected, for example, from the group consisting of glucocorticoids, mTOR inhibitors, cytostatics, antimetabolites, monoclonal antibodies, anti-T-lymphocyte globulin, mycophenolates, and combinations, more particularly mixtures, of at least two of the immunosuppressants.

mTOR inhibitors may be selected, for example, from the group consisting of sirolimus, temsirolimus, everolimus, rapamycin, and combinations, more particularly mixtures, of at least two of the mTOR inhibitors.

Cytostatics may be selected, for example, from the group consisting of alkylating substances, cyclophosphamides, and combinations, more particularly mixtures, of at least two of the cytostatics.

The at least one modulator that modulates blood cells, more particularly immune cells, may be selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the kit is used in vitro for diagnostics, more particularly therapy-accompanying diagnostics, relating to a transplant, more particularly for the prognostic assessment of the prospects of success and/or predicting a clinical outcome of a transplant and/or determining the suitability and/or effectiveness and/or dose of compounds, more particularly immunosuppressants, for a transplant, and/or therapy monitoring of a transplant.

The expression “checkpoint inhibitors” means compounds that inhibit so-called immune checkpoints. The expression “immune checkpoints” means receptors on the membrane of T lymphocytes that attenuate (anti-inflammatory immune checkpoints) or heighten (proinflammatory immune checkpoints) the immune response thereof.

The checkpoint inhibitors may be selected, for example, from the group consisting of CTLA4 inhibitors, PD-1 inhibitors, PD-L1 inhibitors, OX40 inhibitors, 4-1BB inhibitors, TIM3 inhibitors, antibodies to CTLA4, antibodies to PD-1, antibodies to PD-L1, antibodies to OX40, antibodies to 4-1BB, antibodies to TIM3, and combinations, more particularly mixtures, of at least two of the checkpoint inhibitors.

CTLA-4 inhibitors may be selected, for example, from the group consisting of ipilimumab, tremelimumab, and combinations, more particularly mixtures, thereof.

PD-1 inhibitors may be selected, for example, from the group consisting of cemiplimab, nivolumab, pembrolizumab, spartalizumab, and combinations, more particularly mixtures, thereof.

PD-L1 inhibitors may be selected, for example, from the group consisting of atezolizumab, avelumab, durvalumab and combinations, more particularly mixtures, thereof.

Calcineurin inhibitors may be selected, for example, from the group consisting of ciclosporin A, tacrolimus, pimecrolimus, and combinations, more particularly mixtures, of at least two of the calcineurin inhibitors.

DMARDs may be selected, for example, from the group consisting of methotrexate, azathioprine, leflunomide, sulfasalazine, chloroquine, calcineurin inhibitors, and combinations, more particularly mixtures, of at least two of the DMARDs.

The above-described examples relating to diagnostics, more particularly therapy-accompanying diagnostics, in a transplant have in particular the advantages hereinbelow.

It is possible, even before performing a transplant, to check the compatibility of cells and/or antigens of a transplant donor and/or compounds, more particularly immunosuppressants, with the immune system of a transplant recipient. The risk of occurrence of rejection reactions in vivo can thus advantageously be minimized even before surgery. In addition, it is, for example, particularly advantageous to check which compounds, more particularly immunosuppressants, are for a defined recipient—in the sense of personalized medicine—the most promising or most effective or have the lowest risk in respect of a transplant having the best-possible outcome. In addition, it is after a transplant possible to check whether, for example, a compound selected initially, i.e., before the transplant had been performed, more particularly an immunosuppressant selected initially, and/or an initially selected dosage of a compound, more particularly of an immunosuppressant, is capable of/sufficient to achieve the desired therapeutic effect, more particularly immunological suppression, in vivo, i.e., in a transplant recipient. What is critical for the transplant recipient is that the dosage of the employed compound or combinations of a plurality of compounds is neither too low nor too high, since otherwise this can result, in the former example, in rejection of the transplanted organ or, in the latter example, in life-threatening infections or the development of tumors. We make it possible in particular to be able to carry out such complex, technically difficult investigations even outside specialist laboratories and by personnel (doctors, nurses, medical assistants) with no experience of cell cultures, and also within short time frames. The latter in particular is an essential precondition for relevant and informative therapy control, particularly in transplants.

The at least one activator that activates or stimulates blood cells, more particularly immune cells, may be selected from the group consisting of autoantigens, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the kit is used in vitro for diagnostics, more particularly therapy-accompanying diagnostics, relating to an autoimmune disease, more particularly for characterizing the stage of an autoimmune disease and/or for the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease and/or for therapy monitoring of an autoimmune disease.

The expression “autoantigens” means endogenous molecules, for example, surface structures of cell membranes, more particularly surface structures of tissue cells and/or immune cells, or intracellular components, more particularly components of the cytoplasm and/or of the cell organelles and/or of the cell nucleus, that act as antigens in autoimmunity.

The autoantigens may be modified, more particularly citrullinated and/or ubiquitinated.

The expression “citrullinated autoantigens” means autoantigens in which the amino acid arginine has been converted into the amino acid citrulline.

The expression “ubiquitinated antigens” means autoantigens having a ubiquitin residue.

The autoantigens may be selected, for example, from the group consisting of myelin basic protein (MBP), outer surface protein (OspA), leukocyte function-associated antigen 1α (LFA-1α), acetylcholinesterase receptor antigen (AChR), and combinations, more particularly mixtures, of at least two of the autoantigens.

With regard to activators that activate or stimulate blood cells, more particularly immune cells, mentioned in the abovementioned example, reference is made in full to the corresponding statements made in the context of diagnostics, more particularly therapy-accompanying diagnostics, relating to a transplant. The activators that activate or stimulate blood cells, more particularly immune cells, that are additionally described therein apply also to the abovementioned example.

The at least one inhibitor that inhibits or suppresses blood cells, more particularly immune cells, may be selected from the group consisting of anti-inflammatory compounds/drugs, immunosuppressants, and combinations, more particularly mixtures, of at least two of the inhibitors, when the kit is used in vitro for diagnostics, more particularly therapy-accompanying diagnostics, relating to an autoimmune disease, more particularly for characterizing the stage of an autoimmune disease and/or the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease, and/or therapy monitoring of an autoimmune disease.

With regard to anti-inflammatory compounds/drugs and immunosuppressants, reference is made in full to the corresponding statements made in the context of diagnostics, more particularly therapy-accompanying diagnostics, relating to a transplant. The anti-inflammatory compounds/drugs and immunosuppressants described therein apply also to the abovementioned example.

The at least one modulator that modulates blood cells, more particularly immune cells, may be selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the kit is used in vitro for diagnostics, more particularly therapy-accompanying diagnostics, relating to an autoimmune disease, more particularly for characterizing the stage of an autoimmune disease and/or the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease, and/or therapy monitoring of an autoimmune disease.

With regard to suitable checkpoint inhibitors, calcineurin inhibitors, DMARDs, and TLR agonists, reference is made in full to the corresponding statements made in the context of diagnostics, more particularly therapy-accompanying diagnostics, relating to a transplant. The checkpoint inhibitors, calcineurin inhibitors, DMARDs, and TLR agonists described therein apply also to the abovementioned example.

The above-described examples relating to diagnostics, more particularly therapy-accompanying diagnostics, in an autoimmune disease have in particular the advantages hereinbelow.

Characterization of the stage of the autoimmune disease can particularly advantageously be determined on the basis of a disease-relevant degree of activation of blood cells, more particularly immune cells, preferably T cells and/or monocytes and/or macrophages, present in a whole blood sample. A further advantage is that it is possible to check which compounds, more particularly immunosuppressants, are the most promising or most effective in respect of a therapy of an autoimmune disease having the best-possible outcome.

A further particular advantage is that even during therapy of an autoimmune disease it is possible to check whether, for example, a compound selected initially, i.e., before the therapy had been carried out, more particularly an initially selected immunosuppressant, and/or an initially selected dosage of a compound, more particularly of an immunosuppressant, is capable of/sufficient to achieve and maintain the desired therapeutic effect, more particularly immune-logical suppression, in vivo, i.e., in a patient, or whether controlling of the dosage becomes necessary. What is critical here for the patient is that the dosage of the employed compound or combinations of a plurality of compounds is neither too low nor too high, since otherwise this can result, in the former example, in exacerbation of the autoimmune disease or, in the latter example, in life-threatening infections or the development of tumors. We make it possible in particular to be able to carry out such complex, technically difficult investigations even outside specialist laboratories and by personnel (doctors, nurses, medical assistants) with no experience of cell cultures, and also within short time frames. The latter in particular is an essential precondition for relevant and informative therapy control, particularly in autoimmune diseases.

The at least one activator that activates or stimulates blood cells, more particularly immune cells, may be selected from the group consisting of tumor antigens, tumor vaccines, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the kit is used in vitro for diagnostics, more particularly therapy-accompanying diagnostics, relating to a tumor disease, more particularly for the prognostic assessment of the prospects of success and/or predicting a clinical outcome of a therapy of a tumor disease and/or determining the suitability and/or effectiveness and/or dose of compounds for a therapy of a tumor disease and/or therapy monitoring of a tumor disease.

The tumor antigens may be selected, for example, from the group consisting of MAGE, BAGE, GAGE, CTAG, NY-ESO, MUC-1, CDK4, beta-catenin, and combinations, more particularly mixtures, of at least two of the tumor antigens.

The tumor vaccines may, for example, be combinations of a tumor antigen and an adjuvant, for example, a combination of PAP (tumor antigen) and GMCSF (adjuvant) or a combination of MUC-1 (tumor antigen) and monophosphoryl lipid A (adjuvant).

With regard to activators that activate or stimulate blood cells, more particularly immune cells, mentioned in the abovementioned example, reference is made in full to the corresponding statements made in the context of diagnostics, more particularly therapy-accompanying diagnostics, relating to a transplant. The activators that activate or stimulate blood cells, more particularly immune cells, that are additionally described therein apply also to the abovementioned example.

The at least one modulator that modulates blood cells, more particularly immune cells, may be selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the kit is used in vitro for diagnostics, more particularly therapy-accompanying diagnostics, relating to a tumor disease, more particularly for the prognostic assessment of the prospects of success and/or predicting a clinical outcome of a therapy of a tumor disease and/or determining the suitability and/or effectiveness and/or dose of compounds for a therapy of a tumor disease and/or therapy monitoring of a tumor disease.

With regard to suitable checkpoint inhibitors, calcineurin inhibitors, DMARDs, and TLR agonists, reference is made in full to the corresponding statements made in the context of diagnostics, more particularly therapy-accompanying diagnostics, relating to a transplant. The checkpoint inhibitors, calcineurin inhibitors, DMARDs, and TLR agonists described therein apply also to the abovementioned example.

The above-described examples relating to diagnostics, more particularly therapy-accompanying diagnostics, in a tumor disease have in particular the advantages hereinbelow.

A particular advantage is that it is possible to check which compounds are the most promising or most effective in respect of a therapy of a tumor disease having the best-possible outcome. A further advantage is that even during therapy of a tumor disease it is possible to check whether, for example, a compound selected initially, i.e., before the therapy had been carried out, and/or an initially selected dosage of a compound is capable of/sufficient to achieve the desired therapeutic effect in vivo, i.e., in a patient. This is of particularly great importance because (a) tumor cells over the course of time show changes (usually due to further mutations) in their response to certain therapeutic agents, (b) the addition of further drugs alters the response to the compounds initially selected, (c) certain drugs are after some time metabolized or excreted more rapidly (due, e.g., to induction of the liver enzymes responsible for metabolization), or (d) through upregulation of multidrug resistance genes.

The at least one activator that activates or stimulates blood cells, more particularly immune cells, may be selected from the group consisting of antigens, viral antigens, bacterial antigens, parasite antigens, autoantigens, tumor antigens, and combinations, more particularly mixtures, of at least two of the activators.

The antigens may be selected, for example, from the group consisting of viral antigens, bacterial antigens, parasite antigens, autoantigens, tumor antigens, experimental activators for immune cells, and combinations, more particularly mixtures, of at least two of the antigens.

The viral antigens may be selected, for example, from the group consisting of adenovirus antigens, dengue virus antigens, hepatitis B virus antigens, hepatitis A virus antigens, TBE virus antigens, yellow fever virus antigens, HPV virus antigens, influenza virus antigens, measles virus antigens, mumps virus antigens, smallpox virus antigens, polio virus antigens, rotavirus antigens, rubella virus antigens, rabies virus antigens, and combinations, more particularly mixtures, of at least two of the viral antigens.

The bacterial antigens may be selected, for example, from the group consisting of Bacillus anthracis antigens, BCG (Bacillus Calmette-Guérin) antigens, Haemophilus antigens, typhus pathogen antigens, diphtheria pathogen antigens, spotted fever pathogen antigens, tetanus pathogen antigens, meningitis pathogen antigens, whooping cough pathogen antigens, plague pathogen antigens, pneumococcus antigens, and combinations, more particularly mixtures, of at least two of the bacterial antigens.

The parasite antigens may be selected from the group consisting of hookworm antigens, trypanosome antigens, schistosome antigens, and combinations, more particularly mixtures, of at least two of the parasite antigens.

The expression “experimental activators for immune cells” means all of the previously mentioned classes of activators.

The experimental immune cell activators may, for example, be components of bacteria, viruses, fungi or analogs thereof, for example, endotoxins, lipopolysaccharides, superantigens, nucleic acid fragments, polysaccharides, proteoglycans, lipoteichoic acids, cytokines, ligands of various activating receptors (including activating antibodies to surface receptors) or combinations, more particularly mixtures, of at least two of the experimental activators for immune cells. With regard to further features and advantages of the compound classes mentioned in this paragraph, reference is made in full to the prior description.

With regard to autoantigens and tumor antigens mentioned, reference is made in full to the autoantigens and tumor antigens disclosed in the context of diagnostics, more particularly therapy-accompanying diagnostics, relating to a transplant and an autoimmune disease, which accordingly apply also to the abovementioned example.

The at least one modulator that modulates blood cells, more particularly immune cells, may be selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the kit is used in vitro for diagnostics, more particularly therapy-accompanying diagnostics, relating to a vaccination, more particularly for the prognostic assessment of the prospects of success and/or predicting a clinical outcome of a vaccination and/or therapy monitoring of a vaccination.

With regard to suitable checkpoint inhibitors, calcineurin inhibitors, DMARDs, and TLR agonists, reference is made in full to the corresponding statements made in the context of diagnostics, more particularly therapy-accompanying diagnostics, relating to a transplant. The checkpoint inhibitors, calcineurin inhibitors, DMARDs, and TLR agonists described therein apply also to the abovementioned example.

The kit may also include at least one co-activator.

The expression “co-activator” means a compound capable of promoting and/or enhancing the activating effect of the at least one activator that activates or stimulates blood cells, more particularly immune cells, more particularly the activating or stimulating effect thereof on the secretion of cytokines by blood cells present in a whole blood sample, more particularly immune cells. The co-activator may also be referred to as a co-stimulant.

The expression “co-activator” may mean just one co-activator type or a combination, more particularly a mixture, of different co-activator types.

The at least one co-activator is preferably selected from the group consisting of ligands (such as natural and/or semisynthetic and/or synthetic ligands) of CD (cluster of differentiation) molecules, antibodies (such as agonistic and/or antagonistic antibodies) to CD (cluster of differentiation) molecules, TLR ligands, NOD ligands, cytokines, and combinations, more particularly mixtures, of at least two of the co-activators.

For example, the co-activator may be a ligand (such as natural and/or semisynthetic and/or synthetic ligands) of CD28, CD40, CD40L, CD80, CD86 or CD278.

In addition, the co-activator may, for example, be an antibody (such as agonistic and/or antagonistic antibodies) to CD28, CD40, CD40L, CD80, CD86 or CD278.

The TLR ligands may be selected, for example, from the group consisting of lipopolysaccharides, endotoxins, R848, gardiquimod, poly-I:C, ssRNA, CpG oligonucleotides, lipoproteins, lipoteichoic acids, flagellin, zymosan, and combinations, more particularly mixtures, of at least two of the TLR ligands.

The NOD ligands may be selected from the group consisting of MDP, iE-DAP, CpG, derivatives of the NOD ligands, and combinations, more particularly mixtures, of at least two of the NOD ligands.

The kit may also include at least one co-inhibitor.

The expression “co-inhibitor” means more particularly a compound capable of promoting and/or enhancing the inhibitory or suppressive effect of the at least one inhibitor that inhibits or suppresses blood cells, more particularly immune cells, more particularly the inhibitory or suppressive effect thereof on the secretion of cytokines by blood cells present in a whole blood sample, more particularly immune cells. The co-inhibitor may also be referred to as a co-suppressant.

The expression “at least one co-inhibitor” may just one co-inhibitor type or a combination, more particularly a mixture, of different co-inhibitor types.

The at least one co-inhibitor is preferably selected from the group consisting of ligands of CD (cluster of differentiation) molecules, antibodies to CD (cluster of differentiation) molecules, and combinations, more particularly mixtures, thereof.

The antibodies to CD (cluster of differentiation) surface proteins may be selected from the group consisting of agonistic antibodies to CD223 (LAG-3), agonistic antibodies to

CD279, agonistic antibodies to CD152, and combinations, more particularly mixtures, of at least two of the antibodies to CD (cluster of differentiation) surface proteins.

The ligands of CD (cluster of differentiation) surface proteins may be selected from the group consisting of ligands of CD223 such as FGL-1, ligands of CD279 such as CD274, ligands of CD152 such as CD80 and/or CD86, and combinations, more particularly mixtures, of at least two of the ligands of CD (cluster of differentiation) surface proteins.

The kit may also include at least one co-modulator.

The expression “co-modulator” means a compound or a cell capable of promoting and/or enhancing the modulating, i.e., the concentration- and/or environment-dependent activating or inhibitory effect of the at least one modulator.

The expression “at least one co-modulator” may mean just one co-modulator type or a combination, more particularly a mixture, of different co-modulator types.

The at least one co-modulator is preferably selected from the group consisting of inhibitors of enzymes of tryptophan degradation such as TDO and/or IDO, modulating metabolites, ligands, antibodies of receptors, inhibitors or allosteric regulators of enzymes, more particularly of enzymes from signal transduction cascades, inhibitors or activators of ion channels, and combinations, more particularly mixtures, of at least two of the co-modulators.

The transplant may be an organ transplant, more particularly a kidney transplant, liver transplant, heart transplant, lung transplant, pancreas transplant, small intestine transplant, bone marrow transplant, bone transplant or skin transplant.

The autoimmune disease may be alopecia areata, autoimmune enteropathy, autoimmune hepatitis, APECED, bullous pemphigoid, chronic type A gastritis, eosinophilic granulomatosis with polyangiitis, CIDP, chronic active hepatitis, ulcerative colitis, dermatomyositis, type 1 diabetes mellitus, dermatitis herpetiformis (Duhring's disease), endocrine orbitopathy, epidermolysis bullosa acquisita, glomerulonephritis, Goodpasture syndrome, granulomatosis with polyangiitis, Guillain-Barré syndrome, Hashimoto's thyroiditis, idiopathic thrombocytopenic purpura, lichen sclerosus, lichen mucosae, linear IgA dermatosis, lupus erythematosus, microscopic polyangiitis, Graves' disease, Behçet's disease, Crohn's disease, ankylosing spondylitis, multiple sclerosis, myasthenia gravis, narcolepsy, PANDAS, pemphigus foliaceus, pemphigus seborrhoicus, pemphigus vulgaris, polychondritis, polymyalgia rheumatica, polymyositis, psoriasis, primary biliary cirrhosis or primary biliary cholangitis, primary chronic polyarthritis, rheumatic fever, rheumatoid arthritis, giant cell arteritis, SAPHO syndrome, sarcoidosis (Boeck's disease), Sjögren's syndrome, scleroderma, stiff-person syndrome, sympathetic ophthalmia, systemic lupus erythematosus, Henoch-Schönlein purpura, Wegener's granulomatosis, vitiligo or celiac disease.

The tumor disease may be a carcinoma, a melanoma, a blastoma, a lymphoma, a leukemia or a sarcoma.

The carcinoma is preferably selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, rectal carcinoma, laryngeal carcinoma, esophageal carcinoma, gastric carcinoma, breast carcinoma, kidney carcinoma, ovarian carcinoma, pancreatic carcinoma, pharyngeal carcinoma, oropharyngeal carcinoma, prostate carcinoma, thyroid carcinoma and cervical carcinoma.

The sarcoma may be selected from the group consisting of angiosarcoma, chondrosarcoma, Ewing's sarcoma, fibrosarcoma, Kaposi's sarcoma, liposarcoma, leiomyosarcoma, malignant fibrous histiocytoma, neurogenic sarcoma, osteosarcoma and rhabdomyosarcoma.

The vaccination may be an antiviral vaccination, antibacterial vaccination, antiparasitic vaccination, anti-autoimmunological vaccination or antitumor vaccination.

The at least one activator that activates or stimulates blood cells, more particularly immune cells, and/or the at least one inhibitor that inhibits or suppresses blood cells, more particularly immune cells, and/or the at least one modulator that modulates or influences blood cells, more particularly immune cells, and/or the at least one co-activator and/or the at least one co-inhibitor and/or the at least one co-modulator may be present in liquid, especially dissolved, form.

At least some of the components, more particularly all of the components, of the kit may be present spatially or physically separate from one another.

The at least one activator that activates or stimulates blood cells, more particularly immune cells, and/or the at least one inhibitor that inhibits or suppresses blood cells, more particularly immune cells, and/or the at least one modulator that modulates or influences blood cells, more particularly immune cells, and/or the at least one co-activator and/or the at least one co-inhibitor and/or the at least one co-modulator may be present in the blood collection container. Alternatively, the at least one activator that activates or stimulates blood cells, more particularly immune cells, and/or the at least one inhibitor that inhibits or suppresses blood cells, more particularly immune cells, and/or the at least one modulator that modulates or influences blood cells, more particularly immune cells, and/or the at least one co-activator and/or the at least one co-inhibitor and/or the at least one co-modulator may be present spatially or physically separate from the blood collection container.

The nutrient medium may be present in the blood collection container. Alternatively, the nutrient medium may be present spatially or physically separate from the blood collection container.

The blood collection container, more particularly the syringe, may have a hollow cylinder and a closure cap, more particularly a screw cap, for the hollow cylinder. The hollow cylinder may be circular in cross section, i.e., it may be circular-cylindrical in design. Alternatively, the hollow cylinder may be non-circular, more particularly polygonal such as square or hexagonal, in cross section. A plunger, more particularly a syringe plunger, having a plunger rod is preferably associated with the hollow cylinder. Particularly preferably, the plunger, more particularly syringe plunger, is arranged such that the plunger rod can be displaced in the hollow cylinder to create a seal. The plunger rod may have a defined break point. This allows the plunger rod to be snapped off once blood, more particularly whole blood, has been transferred into the hollow cylinder, which makes the handling of the syringe easier, for example, in respect of subsequent incubation. The closure cap may more particularly be a screw cap. The closure cap preferably has an attachment socket for a cannula, more particularly a butterfly cannula. The attachment socket preferably has a seal, more particularly a seal that may be pierced by a cannula, more particularly a butterfly cannula. The seal may be made, for example, of rubber or of another elastic or self-sealing material.

The blood collection set or blood collection system may comprise, in addition to the syringe, a cannula, more particularly a butterfly cannula, and/or blood collection tubing, more particularly transparent blood collection tubing, for example, made of a flexible plastic such as polypropylene, polyethylene or silicone.

The kit may also comprise a component selected from the group consisting of anticoagulant, thermostatically controllable device such as a thermoblock, a number of cooling blocks, a refrigerator, a freezer, a container with dry ice, a nitrogen tank, and combinations of at least two of the components. The thermostatically controllable device is more particularly provided as a means of setting, in the blood collection container, a suitable incubation temperature for the incubation of blood, more particularly of whole blood, of the nutrient medium and of the at least one activator that activates or stimulates blood cells, more particularly immune cells, and/or of the at least one inhibitor that inhibits or suppresses blood cells, more particularly immune cells. The number of cooling blocks and/or the freezer and/or the container with dry ice and/or the nitrogen tank are/is more particularly provided for the cooling of the contents of the blood collection container after incubation, for example, during storage and/or transport of the blood collection container, and/or of samples obtained therefrom.

The kit may also comprise a separating component associated with the blood collection container for the separation, more particularly the spatial separation, of a supernatant and sediment that is being or has been formed in the blood collection container. The separating component can advantageously be inserted into the blood collection container more particularly after a closure cap that seals the blood collection container has been removed. The separating component may advantageously include a pusher.

The separating component may have a valve, more particularly a closable valve, or a valve plunger having a valve, more particularly having a closable valve. In addition, the separating component preferably has a pusher. The valve is particularly preferably open when the valve or the valve plunger is connected to the pusher, and the valve is particularly preferably closed when the valve or the valve plunger are separated from one another.

The examples described in the two previous sections mean there is no need for a centrifugation to separate the supernatant and sediment, which is a particular advantage.

With regard to further features and advantages of the kit as such reference is made to WO 00/08464 A2, the disclosure content of which concerning the kit described therein is incorporated into the content of this disclosure by reference.

We also provide for the in vitro use of a method

for diagnostics, more particularly therapy-accompanying diagnostics, relating to a transplant, more particularly for the prognostic assessment of the prospects of success and/or predicting a clinical outcome of a transplant and/or determining the suitability and/or effectiveness and/or dose of compounds, more particularly immunosuppressants, for a transplant, and/or therapy monitoring of a transplant,

and/or

for diagnostics, more particularly therapy-accompanying diagnostics, relating to an autoimmune disease, more particularly for characterizing the stage of an autoimmune disease and/or the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease, and/or therapy monitoring of an autoimmune disease,

and/or

for diagnostics, more particularly therapy-accompanying diagnostics, relating to a tumor disease, more particularly for the prognostic assessment of the prospects of success and/or predicting a clinical outcome of a therapy of a tumor disease and/or determining the suitability and/or effectiveness and/or dose of compounds for a therapy of a tumor disease and/or therapy monitoring of a tumor disease,

and/or

for diagnostics, more particularly therapy-accompanying diagnostics, relating to a vaccination, more particularly for the prognostic assessment of the prospects of success and/or predicting a clinical outcome of a vaccination and/or therapy monitoring of a vaccination, wherein the method comprises:

a) preparing a mixture comprising whole blood, a nutrient medium for whole blood, and at least one activator that activates or stimulates blood cells, more particularly immune cells, in addition to any activators present in the whole blood, and/or at least one inhibitor that inhibits or suppresses blood cells, more particularly immune cells, in addition to any inhibitors present in the whole blood, and/or at least one modulator that modulates or influences blood cells, more particularly immune cells, in addition to any modulators present in the whole blood,
b) incubating the prepared mixture,
c) separating a messenger-substance-containing supernatant formed through incubation of the prepared mixture from a cell-containing, i.e., blood-cell-containing, more particularly immune-cell-containing, sediment formed through incubation of the prepared mixture, or separating a messenger-substance-containing supernatant formed through centrifugation of the incubated prepared mixture from a cell-containing, i.e., blood-cell-containing, more particularly immune-cell-containing, sediment formed through the centrifugation of the incubated prepared mixture, and
d) detecting and/or determining messenger substances present in the supernatant and/or detecting and/or determining cell surface molecules and/or intracellular activation markers of cells, i.e., blood cells, more particularly immune cells, present in the sediment.

The expression “cell surface molecules” means molecules localized on the surface of cells, i.e., of blood cells, more particularly immune cells. The cell surface molecules may, for example, be cell adhesion molecules (so-called adhesins), receptors such as G-protein-coupled receptors, ion channels, transport molecules such as transport proteins, receptor tyrosine kinases (RTKs), receptor-like protein tyrosine phosphatases (RPTPs), proteoglycans such as syndecan, glycolipids such as gangliosides, CD markers, surface antigens or combinations, more particularly mixtures, of at least two of the surface molecules.

The expression “intracellular activation markers” generally means molecules that undergo characteristic changes after activation or inhibition, either by increasing/decreasing their concentration within cells or by molecular changes, more particularly phosphorylation or dephosphorylation, or else by migrating from one location in the cell to another in a characteristic manner, for example, from the cell membrane or the cytoplasm to the cell nucleus.

Intracellular activation markers may, for example, be receptors, phosphorylated proteins, dephosphorylated proteins, mRNA, cytokines, gene-regulating elements, cAMP, calcium, and combinations, more particularly mixtures, of at least two of the activation markers.

The at least one activator that activates or stimulates blood cells, more particularly immune cells, is preferably selected from the group consisting of cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the method is used in vitro for diagnostics, more particularly therapy-accompanying diagnostics, relating to a transplant, more particularly the prognostic assessment of the prospects of success and/or predicting a clinical outcome of a transplant and/or determining the suitability and/or effectiveness and/or dose of compounds, more particularly immunosuppressants, for a transplant, and/or therapy monitoring of a transplant.

In addition, the at least one inhibitor that inhibits or suppresses blood cells, more particularly immune cells, is preferably selected from the group consisting of anti-inflammatory compounds/drugs, immunosuppressants, and combinations, more particularly mixtures, of at least two of the inhibitors, when the method is used in vitro for diagnostics, more particularly therapy-accompanying diagnostics, relating to a transplant, more particularly for the prognostic assessment of the prospects of success and/or predicting a clinical outcome of a transplant and/or determining the suitability and/or effectiveness and/or dose of compounds, more particularly immunosuppressants, for a transplant, and/or therapy monitoring of a transplant.

In addition, the at least one modulator that modulates blood cells, more particularly immune cells, is preferably selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the method is used in vitro for diagnostics, more particularly therapy-accompanying diagnostics, relating to a transplant, more particularly for the prognostic assessment of the prospects of success and/or predicting a clinical outcome of a transplant and/or determining the suitability and/or effectiveness and/or dose of compounds, more particularly immunosuppressants, for a transplant, and/or therapy monitoring of a transplant.

In addition, the at least one activator that activates or stimulates blood cells, more particularly immune cells, is preferably selected from the group consisting of autoantigens, cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the method is used in vitro for diagnostics, more particularly therapy-accompanying diagnostics, relating to an autoimmune disease, more particularly for characterizing the stage of an autoimmune disease and/or the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease and/or therapy monitoring of an autoimmune disease.

In addition, the at least one inhibitor that inhibits or suppresses blood cells, more particularly immune cells, is preferably selected from the group consisting of anti-inflammatory compounds/drugs, immunosuppressants, and combinations, more particularly mixtures, of at least two of the inhibitors, when the method is used in vitro for diagnostics, more particularly therapy-accompanying diagnostics, relating to an autoimmune disease, more particularly for characterizing the stage of an autoimmune disease and/or the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease and/or therapy monitoring of an autoimmune disease.

In addition, the at least one modulator that modulates blood cells, more particularly immune cells, is preferably selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the method is used in vitro for diagnostics, more particularly therapy-accompanying diagnostics, relating to an autoimmune disease, more particularly for characterizing the stage of an autoimmune disease and/or the prognostic assessment of the prospects of success and/or predicting a clinical outcome of a therapy of an autoimmune disease and/or determining the suitability and/or effectiveness and/or dose of compounds, more particularly immunosuppressants, for a therapy of an autoimmune disease, and/or therapy monitoring of an autoimmune disease.

In addition, the at least one activator that activates or stimultes blood cells, more particularly immune cells, is preferably selected from the group consisting of tumor vaccines, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the method is used in vitro for diagnostics, more particularly therapy-accompanying diagnostics, relating to a tumor disease, more particularly for the prognostic assessment of the prospects of success and/or predicting a clinical outcome of a therapy of a tumor disease and/or of the for suitability and/or effectiveness and/or dose of compounds a therapy of a tumor disease and/or for therapy monitoring of a tumor disease.

In addition, the at least one modulator that modulates blood cells, more particularly immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the method is used in vitro for diagnostics, more particularly therapy-accompanying diagnostics, relating to a tumor disease, more particularly for the prognostic assessment of the prospects of success and/or predicting a clinical outcome of a therapy of a tumor disease and/or of the suitability and/or effectiveness and/or dose of compounds for a therapy of a tumor disease and/or therapy monitoring of a tumor disease. In addition, the at least one activator that activates or stimulates blood cells, more particularly immune cells, is preferably selected from the group consisting of antigens, viral antigens, bacterial antigens, parasite antigens, autoantigens, tumor antigens, and combinations, more particularly mixtures, of at least two of the activators, when the method is used in vitro for diagnostics, more particularly therapy-accompanying diagnostics, relating to a vaccination, more particularly for the prognostic assessment of the prospects of success and/or predicting a clinical outcome of a vaccination and/or therapy monitoring of a vaccination.

In addition, the at least one modulator that modulates blood cells, more particularly immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the method is used in vitro for diagnostics, more particularly therapy-accompanying diagnostics, relating to a vaccination, more particularly for the prognostic assessment of the prospects of success and/or predicting a clinical outcome of a vaccination and/or therapy monitoring of a vaccination.

Step a) and/or step b) and/or step c) and/or step d) are/is preferably carried out in a blood collection vessel, especially in a syringe or a small glass tube, especially in an evacuated small glass tube having a rubber seal. Step b) is preferably carried out over a period of from 1 h to 96 h, more particularly 2 h to 96 h, more particularly 6 h to 72 h, preferably 24 h to 48 h.

In addition, step b) is preferably carried out at a temperature of from 35° C. to 39° C., more particularly 36° C. to 38° C., preferably 37° C.

For the performance of step c), preference is given to using a separating component associated with the blood collection container that has a valve and a pusher or a valve plunger that has a valve and a pusher. Advantageously, the separating component is for this purpose, more particularly after a closure cap sealing the blood collection container has been removed, introduced from above into the blood collection container and pressed into the supernatant, wherein the supernatant flows through the valve or the valve plunger and closes by itself on completion of the pressing-in process and retraction of the pusher so that mixing between the sediment and the supernatant is thereafter no longer possible. The sediment caught in the lower part of the blood collection container, which comprises sedimented, i.e., deposited, blood cells, more particularly immune cells, and the supernatant separated therefrom present in the upper part of the blood collection container can be left in the blood collection container during further handling, for example, until the supernatant is withdrawn for the performance of step d).

For the performance of step d), an immunoassay such as ELISA, Luminex, Simoa analysis or multiplex analyses and/or flow cytometry (for example, to quantify surface markers or the degree of phosphorylation of proteins from signal transduction cascades or the translocation of gene-regulating factors) and/or a nucleic acid diagnostic method (for example, for the detection of mRNAs and/or miRNAs using NanoString or other technologies) is preferably carried out.

With regard to further features and advantages of the in vitro use of our method first apply to the above-described second method. The features and advantages described herein, more particularly in respect of the at least one activator that activates or stimulates blood cells, more particularly immune cells, the at least one inhibitor that inhibits or suppresses blood cells, more particularly immune cells, the at least one modulator for modulating or influencing blood cells, more particularly immune cells, the blood collection container, and the separating component accordingly apply also to the in vitro use of our method according to our second method.

We also provide a messenger-substance-containing supernatant or to a cell-containing, i.e., blood-cell-containing, more particularly immune-cell-containing, sediment for application or use in vitro

in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant,

and/or

in diagnostics, more particularly therapy-accompanying diagnostics, relating to an autoimmune disease, especially in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease,

and/or

in diagnostics, more particularly therapy-accompanying diagnostics, relating to a tumor disease, more particularly in the prognostic assessment of the prospects of success and/or in predicting a clinical outcome of a therapy of a tumor disease and/or in determining the suitability and/or effectiveness and/or dose of compounds for a therapy of a tumor disease, and/or in therapy monitoring of a tumor disease,

and/or

in diagnostics, more particularly therapy-accompanying diagnostics, relating to a vaccination, more particularly in the prognostic assessment of the prospects of success and/or in predicting a clinical outcome of a vaccination and/or in therapy monitoring of a vaccination,

wherein the messenger-substance-containing supernatant or the cell-containing sediment is produced or producible by a method comprising:

a) preparing a mixture comprising whole blood, a nutrient medium for whole blood, and at least one activator that activates or stimulates blood cells, more particularly immune cells, in addition to any activators present in the whole blood, and/or at least one inhibitor that inhibits or suppresses blood cells, more particularly immune cells, in addition to any inhibitors present in the whole blood, and/or at least one modulator that modulates or influences blood cells, more particularly immune cells, in addition to any modulators present in the whole blood,
b) incubating the prepared mixture, and
c) separating a messenger-substance-containing supernatant formed through the incubation of the prepared mixture from a cell-containing, i.e., blood-cell-containing, more particularly immune-cell-containing, sediment formed through the incubation of the prepared mixture, or separating a messenger-substance-containing supernatant formed through the centrifugation of the incubated prepared mixture from a cell-containing, i.e., blood-cell-containing, more particularly immune-cell-containing, sediment formed through the centrifugation of the incubated prepared mixture.

The at least one activator that activates or stimulates blood cells, more particularly immune cells, is preferably selected from the group consisting of cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the messenger-substance-containing supernatant or cell-containing sediment is respectively a messenger-substance-containing supernatant or cell-containing sediment for application or use in vitro in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant.

In addition, the at least one inhibitor that inhibits or suppresses blood cells, more particularly immune cells, is preferably selected from the group consisting of anti-inflammatory compounds/drugs, immunosuppressants, and combinations, more particularly mixtures, of at least two of the inhibitors, when the messenger-substance-containing supernatant or cell-containing sediment is respectively a messenger-substance-containing supernatant or cell-containing sediment for application or use in vitro in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant.

In addition, the at least one modulator that modulates blood cells, more particularly immune cells, is preferably selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the messenger-substance-containing supernatant or cell-containing sediment is respectively a messenger-substance-containing supernatant or cell-containing sediment for application or use in vitro in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant.

In addition, the at least one activator activates or stimulates blood cells, more particularly immune cells, is preferably selected from the group consisting of autoantigens, cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the messenger-substance-containing supernatant or cell-containing sediment is respectively a messenger-substance-containing supernatant or cell-containing sediment for application or use in vitro in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease.

In addition, the at least one inhibitor that inhibits or suppresses blood cells, more particularly immune cells, is preferably selected from the group consisting of anti-inflammatory compounds/drugs, immunosuppressants, and combinations, more particularly mixtures, of at least two of the inhibitors, when the messenger-substance-containing supernatant or cell-containing sediment is respectively a messenger-substance-containing supernatant or cell-containing sediment for application or use in vitro in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease.

In addition, the at least one modulator that modulates blood cells, more particularly immune cells, is preferably selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the messenger-substance-containing supernatant or cell-containing sediment is respectively a messenger-substance-containing supernatant or cell-containing sediment for application or use in vitro in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease.

In addition, the at least one activator that activates or stimulates blood cells, more particularly immune cells, is preferably selected from the group consisting of tumor vaccines, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the messenger-substance-containing supernatant or cell-containing sediment is respectively a messenger-substance-containing supernatant or cell-containing sediment for application or use in vitro in diagnostics, more particularly therapy-accompanying diagnostics, relating to a tumor disease, more particularly in the prognostic assessment of the prospects of success and/or in predicting a clinical outcome of a therapy of a tumor disease and/or in determining the suitability and/or effectiveness and/or dose of compounds for a therapy of a tumor disease, and/or in therapy monitoring of a tumor disease.

In addition, the at least one modulator that modulates blood cells, more particularly immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the messenger-substance-containing supernatant or cell-containing sediment is respectively a messenger-substance-containing supernatant or cell-containing sediment for application or use in vitro in diagnostics, more particularly therapy-accompanying diagnostics, relating to a tumor disease, more particularly in the prognostic assessment of the prospects of success and/or in predicting a clinical outcome of a therapy of a tumor disease and/or in the suitability and/or effectiveness and/or dose of compounds for a therapy of a tumor disease, and/or in therapy monitoring of a tumor disease.

In addition, the at least one activator that activates or stimulates blood cells, more particularly immune cells, is preferably selected from the group consisting of antigens, viral antigens, bacterial antigens, parasite antigens, autoantigens, tumor antigens, and combinations, more particularly mixtures, of at least two of the activators, when the messenger-substance-containing supernatant or cell-containing sediment is respectively a messenger-substance-containing supernatant or cell-containing sediment for application or use in vitro in diagnostics, more particularly therapy-accompanying diagnostics, relating to a vaccination, more particularly in the prognostic assessment of the prospects of success and/or in predicting a clinical outcome of a vaccination and/or in therapy monitoring of a vaccination.

In addition, the at least one modulator that modulates blood cells, more particularly immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the messenger-substance-containing supernatant or cell-containing sediment is respectively a messenger-substance-containing supernatant or cell-containing sediment for application or use in vitro in diagnostics, more particularly therapy-accompanying diagnostics, relating to a vaccination, more particularly in the prognostic assessment of the prospects of success and/or in predicting a clinical outcome of a vaccination and/or in therapy monitoring of a vaccination.

With regard to further features and advantages of the messenger-substance-containing supernatant and of the cell-containing sediment, reference is made in full to the statements made in the context of the prior description. The features and advantages described therein, more particularly in respect of the at least one activator that activates or stimulates blood cells, more particularly immune cells, the at least one inhibitor that inhibits or suppresses blood cells, more particularly immune cells, and the at least one modulator that modulates or influences blood cells, more particularly immune cells, apply by analogy also to the messenger-substance-containing supernatant or the cell-containing sediment for application or use in vitro.

We further provide a combination, more particularly in the form of a mixture, comprising whole blood, a nutrient medium for whole blood, and at least one activator that activates or stimulates blood cells, more particularly immune cells, in addition to any activators present in the whole blood, and/or at least one inhibitor that inhibits or suppresses blood cells, more particularly immune cells, in addition to any inhibitors present in the whole blood, and/or at least one modulator that modulates or influences blood cells, more particularly immune cells, in addition to any modulators present in the whole blood, for application or use in vitro

in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant,

and/or

in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease,

and/or

in diagnostics, more particularly therapy-accompanying diagnostics, relating to a tumor disease, more particularly in the prognostic assessment of the prospects of success and/or in predicting a clinical outcome of a therapy of a tumor disease and/or in determining the suitability and/or effectiveness and/or dose of compounds for a therapy of a tumor disease, and/or in therapy monitoring of a tumor disease,

and/or

in diagnostics, more particularly therapy-accompanying diagnostics, relating to a vaccination, more particularly in the prognostic assessment of the prospects of success and/or in predicting a clinical outcome of a vaccination and/or in therapy monitoring of a vaccination.

The at least one activator that activates or stimulates blood cells, more particularly immune cells, is preferably selected from the group consisting of cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the combination, more particularly mixture, is a combination, more particularly mixture, for application or use in vitro in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant.

In addition, the at least one inhibitor that inhibits or suppresses blood cells, more particularly immune cells, is preferably selected from the group consisting of anti-inflammatory compounds/drugs, immunosuppressants, and combinations, more particularly mixtures, of at least two of the inhibitors, when the combination, more particularly mixture, is a combination, more particularly mixture, for application or use in vitro in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant.

In addition, the at least one modulator that modulates blood cells, more particularly immune cells, is preferably selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the combination, more particularly mixture, is a combination, more particularly mixture, for application or use in vitro in diagnostics, more particularly therapy-accompanying diagnostics, relating to a transplant, more particularly in the prognostic assessment of the prospects of success and/or in predicting a clinical outcome of a transplant and/or in determining the suitability and/or effectiveness and/or dose of compounds, more particularly immunosuppressants, for a transplant, and/or in therapy monitoring of a transplant.

In addition, the at least one activator that activates or stimulates blood cells, more particularly immune cells, is preferably selected from the group consisting of autoantigens, cells from a transplant donor, antigen preparations of cells from a transplant donor, antigens, superantigens, mitogens, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the combination, more particularly mixture, is a combination, more particularly mixture, for application or use in vitro in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease.

In addition, the at least one inhibitor inhibits or suppresses blood cells, more particularly immune cells, is preferably selected from the group consisting of anti-inflammatory compounds/drugs, immunosuppressants, and combinations, more particularly mixtures, of at least two of the inhibitors, when the combination, more particularly mixture, is a combination, more particularly mixture, for application or use in vitro in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease.

In addition, the at least one modulator that modulates blood cells, more particularly immune cells, is preferably selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the combination, more particularly mixture, is a combination, more particularly mixture, for application or use in vitro in diagnostics, more particularly therapy-accompanying diagnostics, relating to an autoimmune disease, more particularly in characterizing the stage of an autoimmune disease and/or in the prognostic assessment of the prospects of success and/or in predicting a clinical outcome of a therapy of an autoimmune disease and/or in determining the suitability and/or effectiveness and/or dose of compounds, more particularly immunosuppressants, for a therapy of an autoimmune disease, and/or in therapy monitoring of an autoimmune disease.

In addition, the at least one activator that activates or stimulates blood cells, more particularly immune cells, is preferably selected from the group consisting of tumor vaccines, ligands (such as natural and/or semisynthetic and/or synthetic ligands) of blood cell surface receptors, more particularly immune cell surface receptors, that have an activating or inhibitory effect on blood cells, more particularly immune cells, ligands (such as natural and/or semisynthetic and/or synthetic ligands) of intracellular receptors of blood cells, more particularly immune cells, that have an activating or inhibitory effect on blood cells, more particularly immune cells, ligands (such as natural and/or semisynthetic and/or synthetic ligands) of ion channels that have an activating or inhibitory effect on blood cells, more particularly immune cells, and are accessible via surface receptors or intracellular receptors of blood cells, more particularly immune cells, ligands (such as natural and/or semisynthetic and/or synthetic ligands) of enzymes that have an activating or inhibitory effect on blood cells, more particularly immune cells, and are accessible via surface receptors or intracellular receptors of blood cells, more particularly immune cells, antibodies (such as agonistic and/or antagonistic antibodies) to blood cell surface receptors, more particularly immune cell surface receptors, that have an activating or inhibitory effect on blood cells, more particularly immune cells, antibodies (such as agonistic and/or antagonistic antibodies) to intracellular receptors of blood cells, more particularly immune cells, that have an activating or inhibitory effect on blood cells, more particularly immune cells, antibodies (such as agonistic and/or antagonistic antibodies) to ion channels that have an activating or inhibitory effect on blood cells, more particularly immune cells, and are accessible via surface receptors or intracellular receptors of blood cells, more particularly immune cells, antibodies (such as agonistic and/or antagonistic antibodies) to enzymes that have an activating or inhibitory effect on blood cells, more particularly immune cells, and are accessible via surface receptors or intracellular receptors of blood cells, more particularly immune cells, adjuvants, and combinations, more particularly mixtures, of at least two of the activators, when the combination, more particularly mixture, is a combination, more particularly mixture, for application or use in vitro in diagnostics, more particularly therapy-accompanying diagnostics, relating to a tumor disease, more particularly in the prognostic assessment of the prospects of success and/or in predicting a clinical outcome of a therapy of a tumor disease and/or in the suitability and/or effectiveness and/or dose of compounds for a therapy of a tumor disease, and/or in therapy monitoring of a tumor disease.

In addition, the at least one modulator that modulates blood cells, more particularly immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the combination, more particularly mixture, is a combination, more particularly mixture, for application or use in vitro in diagnostics, more particularly therapy-accompanying diagnostics, relating to a tumor disease, more particularly in the prognostic assessment of the prospects of success and/or in predicting a clinical outcome of a therapy of a tumor disease and/or in the suitability and/or effectiveness and/or dose of compounds for a therapy of a tumor disease, and/or in therapy monitoring of a tumor disease.

In addition, the at least one activator that activates or stimulates blood cells, more particularly immune cells, is preferably selected from the group consisting of antigens, viral antigens, bacterial antigens, parasite antigens, autoantigens, tumor antigens, and combinations, more particularly mixtures, of at least two of the activators, when the combination, more particularly mixture, is a combination, more particularly mixture, for application or use in vitro in diagnostics, more particularly therapy-accompanying diagnostics, relating to a vaccination, more particularly in the prognostic assessment of the prospects of success and/or in predicting a clinical outcome of a vaccination and/or in therapy monitoring of a vaccination.

In addition, the at least one modulator that modulates blood cells, more particularly immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations, more particularly mixtures, of at least two of the modulators, when the combination, more particularly mixture, is a combination, more particularly mixture, for application or use in vitro in diagnostics, more particularly therapy-accompanying diagnostics, relating to a vaccination, more particularly in the prognostic assessment of the prospects of success and/or in predicting a clinical outcome of a vaccination and/or in therapy monitoring of a vaccination.

With regard to further features and advantages of the combination, reference is made in full to the prior description, in particular to the statements made in the context of our first method. The features and advantages described therein, more particularly in respect of the at least one activator that activates or stimulates blood cells, more particularly immune cells, the at least one inhibitor that inhibits or suppresses blood cells, more particularly immune cells, and the at least one modulator that modulates or influences blood cells, more particularly immune cells, apply by analogy also to the combination for application or use in vitro.

Further features and advantages arise from the following description of preferred examples in the form of figures, an associated description of the figures, and examples. The examples described hereinbelow serve merely for further elucidation, but without restricting the scope of this disclosure.

### BRIEF DESCRIPTION OF THE DRAWINGS

The following is shown schematically in the drawings:

FIG. 1: Dose-response curves of four TLR agonists (stimulated release of IL-6) using a kit; activating active substances developed for tumor therapy (TLR agonists); activation of immune cells from six healthy donors quantified on the basis of release of IL-6 (characteristic cytokine for macrophage activation).

FIG. 2: Result of titration of a checkpoint inhibitor (CPI-1, developed for adjuvant immune cell stimulation in tumor patients) in SEB-stimulated cultures (release of IL-2/four healthy donors) using a kit; IL-2 is here specific for T-cell activation.

FIG. 3a: Dose-response curves of two immunosuppressants developed for autoimmune diseases (donor A) using a kit.

FIG. 3b: Dose-response curves of two immunosuppressants developed for autoimmune diseases (donor B) using a kit. Suppressant active substances developed for autoimmune diseases were used. To investigate the inhibitory effect on immune cells, these were stimulated using a TLR agonist. The results of two healthy donors quantified on the basis of release of various inflammation-relevant cytokines are shown.

FIG. 4a: Influence of various additives on SEB-induced T-cell activity (with release of IFNg) using a kit.

FIG. 4b: Influence of various additives on SEB-induced T-cell activity (release of IL-2) using a kit.

FIG. 4c: Influence of various additives on SEB-induced T-cell activity (release of IL-4) using a kit.

FIG. 4d: Influence of various additives on SEB-induced T-cell activity (release of IL-5) using a kit.

FIG. 4e: Influence of various additives on SEB-induced T-cell activity (release of IL-10) using a kit.

FIGS. 4a-e are exemplary depictions of five cytokines measured in the culture supernatants of whole blood cultures by multiplex analysis after different culture times. Stimulation of T-cells by SEB (=“superantigen”) was however carried out with addition of LPS for activation of monocytes/macrophages or of co-stimulus 1. Superantigens activate T cells like normal antigens, but activate a higher number of T cells at the same time than with standard antigens. FIG. 5: Antigen-specifically induced release of IFNg by the influenza vaccine Fluzone using a kit.

FIG. 5 shows clearly that the release of the messenger substance interferon gamma (IFNg), which is characteristic for T-cell activation, took place almost independently of the dose. FIG. 6: Antigen-specifically induced release of IL-2 by the influenza vaccine Fluzone using a kit.

FIG. 6 showing clearly that the release of the messenger substance interleukin-2 (IL-2), which is characteristic for T-cell activation, took place almost independently of the dose.

FIG. 7: A kit after incubation of whole blood, nutrient medium, and the at least one activator for activating blood cells, more particularly immune cells, and/or the at least one inhibitor for inhibiting blood cells, more particularly immune cells, and/or the at least one modulator for modulating blood cells, more particularly immune cells.

### DETAILED DESCRIPTION

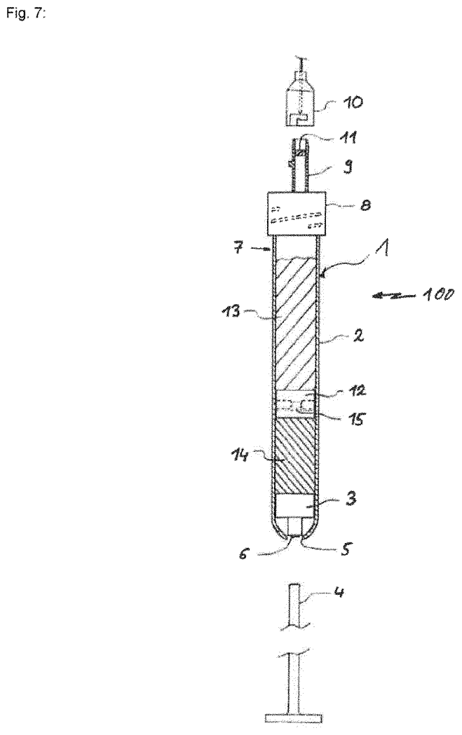

FIG. 7 shows a kit 100.

The kit 100 comprises a blood collection container in the form of a syringe 1, a nutrient medium (not shown), and at least one activator that activates blood cells, more particularly immune cells (not shown) and/or at least one inhibitor that inhibits blood cells, more particularly immune cells (not shown) and/or at least one modulator that modulates blood cells, more particularly immune cells (not shown). With regard to suitable activators/inhibitors/modulators, reference is made in full to the prior description.

The syringe 1 has a hollow cylinder 2 in which a syringe plunger 3 is arranged such that it can be displaced with the aid of a plunger rod 4 to create a seal. The interior of the hollow cylinder 2 is preferably sterile. The hollow cylinder 2 has a taper 5 in the region of the plunger rod 4 that guides the plunger rod 4. The plunger rod 4 has a defined break point 6 close to the syringe plunger 3 that comes to rest in the region of the taper 5 when the plunger is pulled backwards and more particularly enables the plunger rod 4 to be snapped off (easily) when the syringe plunger 3 is guided backwards. The end 7 of the hollow cylinder 2 pointing away from the plunger rod 4 is open over the entire cross section and can be closed with a closure cap, more particularly a screw cap 8. The closure cap 8 preferably has an attachment socket 9 for a cannula or syringe needle 10. The cannula 10 can preferably be locked in place with a bayonet closure. The attachment socket 9 preferably has on the inside a pierceable seal 11, more particularly made of rubber, that is pierced when the cannula 10 is attached and forms a seal again when the cannula 10 is removed. The pierceable seal 11 particularly advantageously makes it possible to ensure the sterility of the interior of the hollow cylinder 2 before, during and after collection of blood.

The kit 100 also includes a separating component, more particularly a valve plunger 12. The separating component 12 is preferably associated with the syringe 1. In the unused state, the separating component 12 is preferably arranged outside the syringe 1. A pusher (not shown in the drawing) is advantageously associated with the separating component 12. After removing the closure cap 8, the pusher can be used to insert the separating component 12 into the syringe 1. Insertion takes place at the end of incubation of a whole blood sample contained in the syringe 1 that has separated into a supernatant 13 and a sediment 14 containing deposited blood cells. For permanent separation of supernatant 13 and sediment 14, the separating component 12 is pushed in as far as the interface between supernatant 13 and sediment 14, wherein the supernatant 13 flows through a valve 15 of the separating component 12 until the separation process is completed on reaching the interface between supernatant 13 and sediment 14. On completion of the pressing-in process and retraction of the pusher of the separating component 12, the valve 15 preferably closes automatically. The syringe plunger 3 can then be tightly resealed by attaching the closure cap 8, and the syringe plunger 3 used as a storage, cooling, and transport container, which means that the blood or separated parts thereof can be stored between blood collection and the actual performance of the detection and/or determination of the cytokines present in the supernatant 13 without any need to be decanted. It is, for example, possible to use, as syringe 1, a syringe commercially available under the name “Monovette” from Sarstedt and, as separating component 12, a valve plunger available under the name “Seraplas” from Sarstedt.

### Examples Section

### Performance of Whole Blood Cultures (General)

Syringes of the kit were frozen immediately after being filled with the appropriate substances dissolved in proprietary nutrient medium and stored at −20° C. until use. After thawing and inverting, a puncture cannula/butterfly or similar hollow needle system was used with the aid of an adapter to puncture a vein of a blood donor/patient. This was followed by the collection of 1 ml of blood by carefully drawing it up directly into the syringe. The contents (blood and nutrient medium with or without additives: activators, inhibitors, modulators or the like) were immediately mixed several times by inverting the syringes and incubated at 37° C. in a block thermostat for the desired culture time of e.g., 24 or 48 hours.

After removing the syringes from the block thermostat at the end of the culture time, the reaction was stopped by carefully inserting a separating valve (“Seraplas filter”), which was accompanied by the permanent separation of the sedimented cells from the culture supernatant.

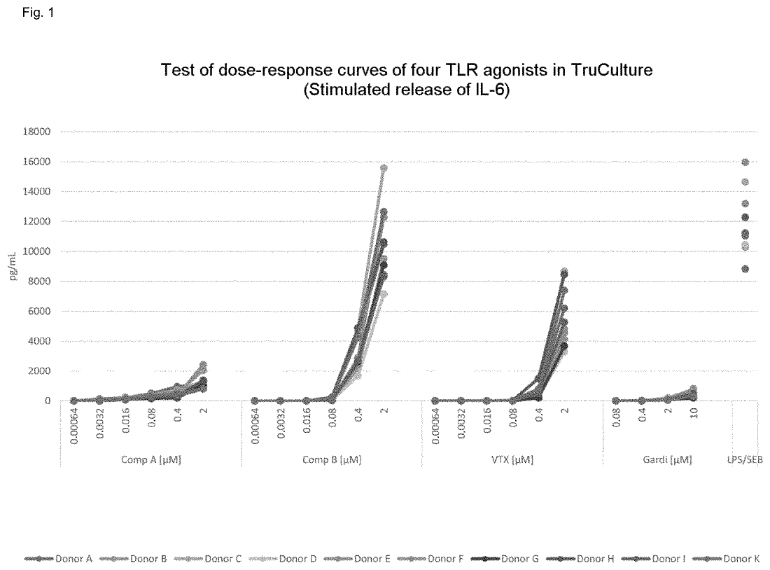

Both the cells and the culture supernatant could then be sent for appropriate methods of detection for determination of the appropriate endpoints. The cells could be investigated to detect intracellular factors, for example, by flow cytometry or RNA analysis, for example, by PCR. For cellular investigations, it could be necessary first to separate the culture supernatant from the cells and then to add to the cells appropriate solutions for stabilization, fixation and/or preservation. The messenger substances released into the culture supernatant could be quantified, for example, by chromatographic methods (for example, HPLC) or by immunoassay such as ELISA, Simoa, Luminex, or other methods. If the cells or the culture supernatant were not analyzed immediately, they could be stored in the syringes at −20° C. until further investigation. Performance of whole blood cultures—Description of the method for the experiment shown in FIG. 1

### Tumor Therapy: Pre-Screening and Therapy Monitoring

The proprietary nutrient medium for these whole blood cultures was supplemented with the TLR activators “compound A,” “compound B,” “VTX” and gardiquimod that were developed for the activation of macrophages in the course of tumor therapies. In addition, a combination of LPS (0.1 μg/ml)+SEB (0.4 μg/ml) was used as a positive control. The concentration of the four TLR activators was in a serial 1:5 dilution between 0.00064 and 2 μM final concentration. The correspondingly freshly loaded syringes were stored at −20° C. until use.

After the syringes had been thawed, they were after venipuncture each filled with 1 ml of blood from the blood donor and incubated as described above at 37° C. in a block thermostat for the desired culture time of 24 hours.

At the end of the incubation, the separating valves were inserted and the syringes stored at −20° C. until testing for cytokine release. IL-6, which is a characteristic cytokine for macrophage activation, was determined as the endpoint by ELISA.

The data presented in FIG. 1 show clear activation of macrophages, the expression of which depended on both the activators and the dosage thereof.

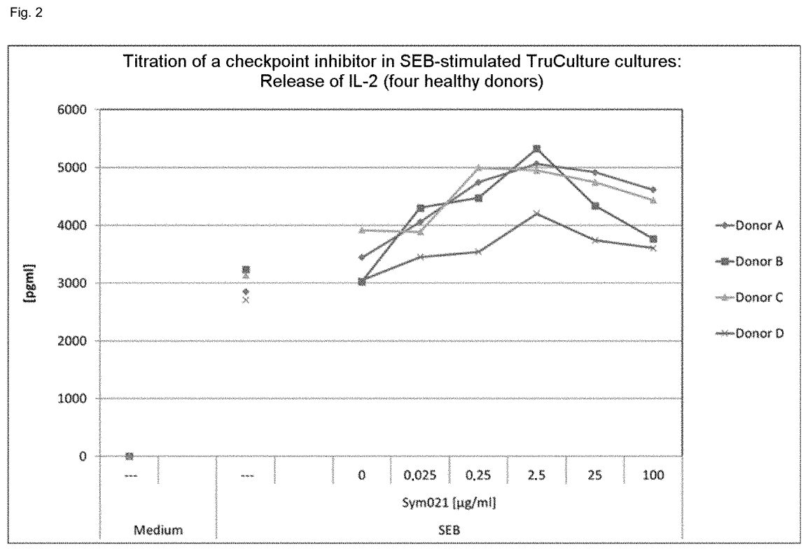

### Performance of Whole Blood Cultures—Description of the Method for the Experiment Shown in FIG. 2

### Tumor Therapy: Pre-Screening and Therapy Monitoring

The proprietary nutrient medium for these whole blood cultures was supplemented with a checkpoint inhibitor developed for the activation of T cells in the course of tumor therapies.

(CPI-1). The activity of such active substances can be demonstrated only in the presence of a T-cell-specific basal stimulus. The superantigen SEB was chosen for this purpose and was also used as a positive control. The correspondingly freshly loaded syringes were stored at −20° C. until use.

After the syringes had been thawed, they were after venipuncture each filled with 1 ml of blood from the blood donor and incubated as described above at 37° C. in a block thermostat for the desired culture time of 48 h.

At the end of the incubation, the separating valves were inserted and the tubes stored at −20° C. until testing for cytokine release. IL-2, which is a characteristic cytokine for T-lymphocyte activation, was determined as the endpoint by ELISA.

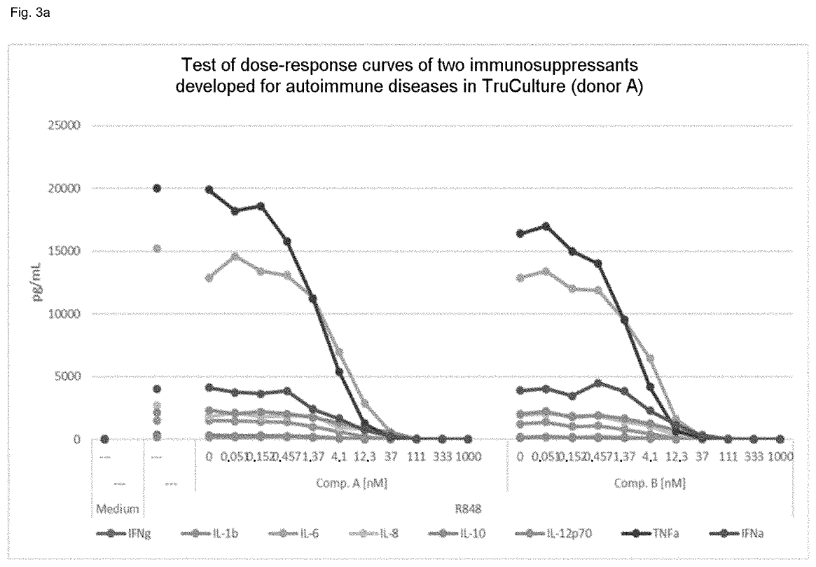

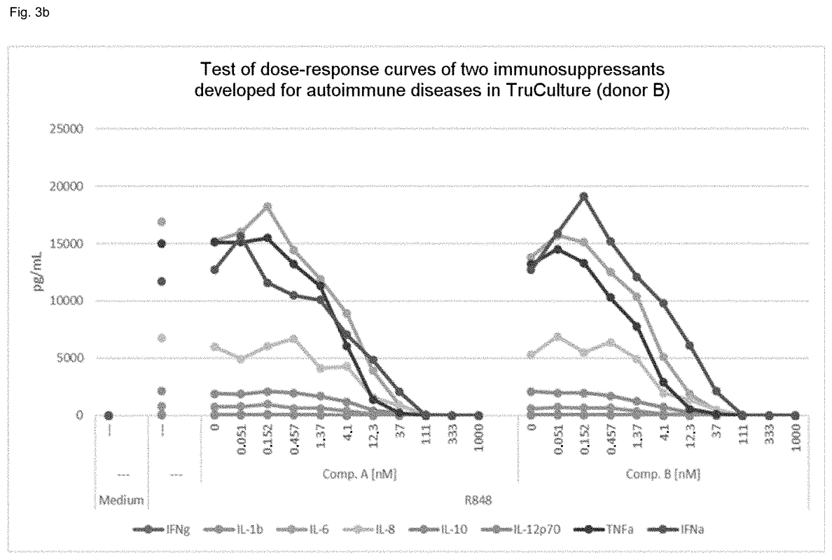

The data presented in FIG. 2 show clear activation of T-lymphocytes, which exhibits a clear improvement in its expression. The strength of the activating effect is moreover individual-specific, as can be seen from the different courses in the cultures of the individual donors. Performance of whole blood cultures—Description of the method for the experiment shown in FIGS. 3a and 3b

### Autoimmune Diseases/Organ Transplantation:

### Individual Pre-Testing of the Suitability of Particular Preparations and Therapy Monitoring

The proprietary nutrient medium for these whole blood cultures was supplemented with the TLR7/TLR8 ligand R848, to induce a relatively broad immune cell response. In addition, two inhibitors newly developed for the treatment of autoimmune diseases (“compound A,” “compound B”) were each added in a dilution series. R848 served as a positive control. The correspondingly freshly loaded syringes were stored at −20° C. until use.

After the syringes had been thawed, they were after venipuncture each filled with 1 ml of blood from the blood donor and incubated as described above at 37° C. in a block thermostat for the desired culture time of 24 hours.

At the end of the incubation, the separating valves were inserted and the tubes stored at −20° C. until testing for cytokine release. A number of different cytokines that are characteristic messenger substances for broader inflammatory responses were determined as the endpoint by Luminex.

The data shown in FIGS. 3a-b reveal very strong, dose-dependent inhibition of various immune cells, with the two compounds showing no appreciable differences. The dose-response curves of the two donors tested were likewise very similar.

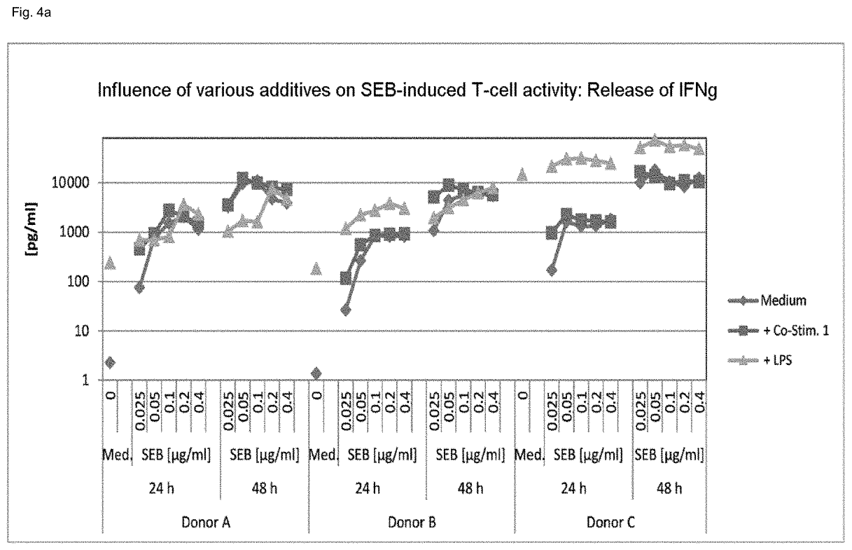

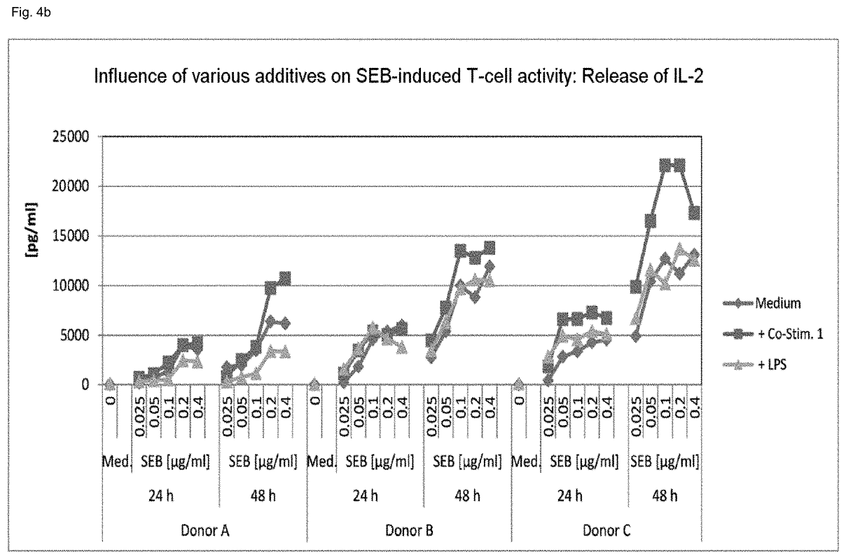

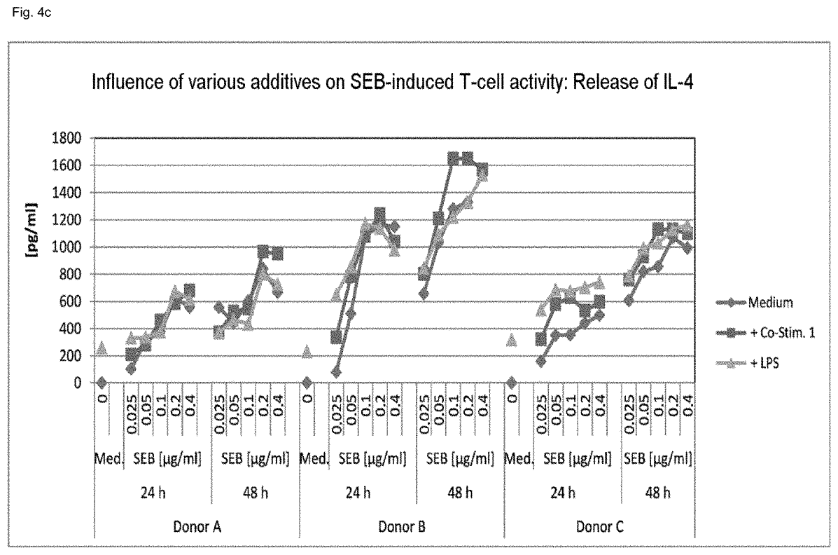

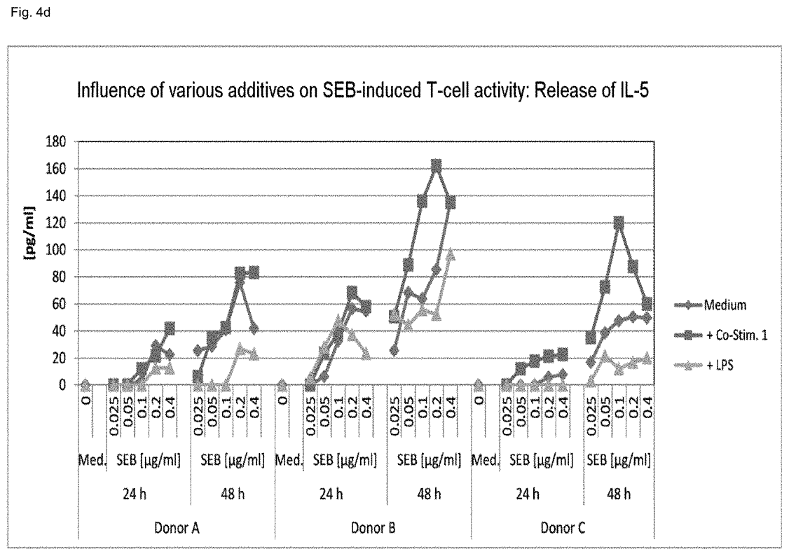

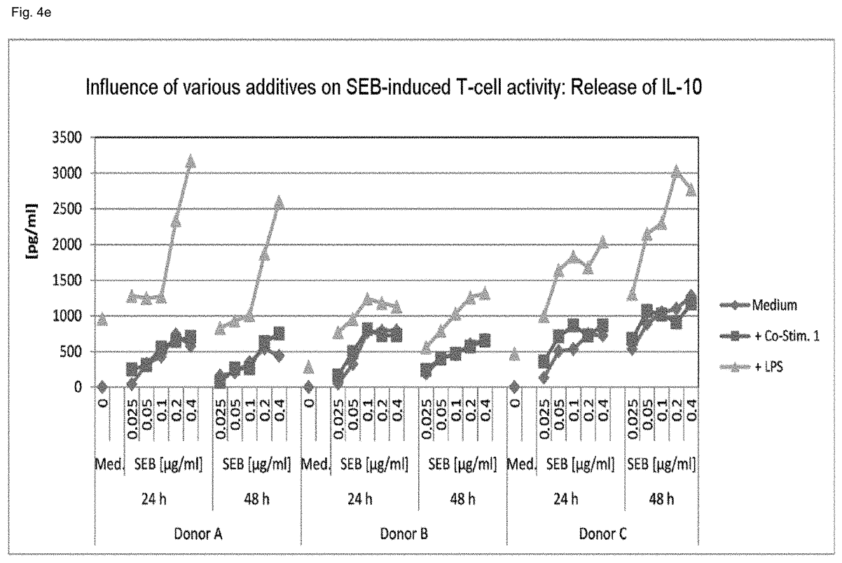

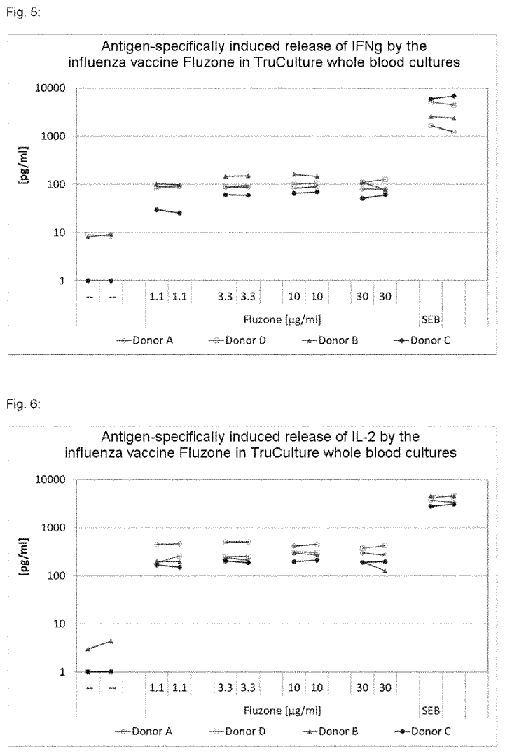

### Performance of Whole Blood Cultures—Description of the Method for the Experiment Shown in FIGS. 4a-e

### Antigen-Specific Responses (Autoimmune Diseases, Vaccinations):

The proprietary nutrient medium for these whole blood cultures was supplemented with the T-cell-specific superantigen SEB, to induce a response in these cells that corresponds to that of normal antigens except that, rather than activating just a few T-lymphocytes, it stimulates the majority of these cells. In addition, two reagents were used (LPS and “Co-Stim. 1,” the latter having been developed in house) to test the extent to which antigen-induced activations can be enhanced. This will be of particular importance when normal antigens are used that, although inducing the same activation pathways in T cells, are able to achieve a response in just a few of these lymphocytes. To control the signal-to-noise ratio of, for example, cytokine assays, it is important in such situations that each individual activatable cell releases as much messenger substance as possible. SEB was titrated to examine whether the co-stimulating agents have different positive effects with different antigen signal strengths. The correspondingly freshly loaded syringes were stored at −20° C. until use.

After the syringes had been thawed, they were after venipuncture each filled with 1 ml of blood from the blood donor and incubated as described above at 37° C. in a block thermostat for the desired culture time of 24 h and 48 h.

At the end of the incubation, the separating valves were inserted and the tubes stored at −20° C. until testing for cytokine release. Five cytokines (IFN-gamma, IL-2, IL-4, IL-5, and IL-10) considered to be relevant in T-cell activations were determined as the endpoint by Luminex.

The data shown in FIGS. 4a-e exhibit clear differences between donors in the overall strength of their immune cell response to SEB stimulation. Clear dose dependencies were in addition observed and this run too demonstrated that T cells benefit from a longer incubation time (48 h). The two co-stimulatory additives, LPS and Co-Stim 1, elicited moderate and also characteristic changes in the cytokine levels of these cultures, which means that further use of these substances may—depending on the situation—be useful. Co-Stim 1 was noteworthy in that it showed clearly enhancing effects for IL-2 and IL-5: IL-2 in respect of activation of Th1 cells and IL-5 in respect of activation of Th2 cells.

### Performance of Whole Blood Cultures—Description of the Method for the Experiment in Tables 1-4

The proprietary nutrient medium for these whole blood cultures was supplemented with individual or combined “recall antigens” (tetanus toxoid, TeTo, or peptides from various prominent viruses such as cytomegalovirus, Epstein Barr, and influenza, CEF) on the one hand, and also with various co-stimulants (Co-Stim. 1, Co-Stim. 2, CM 2, CM 3, and Stim. 2) in various combinations. Whereas the CEF peptides were used in different concentrations, the very good response obtained with tetanus toxoid meant that this could be used with just one final concentration (2 μg/mL). Syringes filled just with medium served as negative controls (Neg. Co). Immediately after filling, the syringes were frozen and stored at −20° C. until use.

After the syringes had been thawed, they were after venipuncture each filled with 1 ml of blood from the blood donor and incubated as described above at 37° C. in a block thermostat for 48 hours.

At the end of the incubation, the separating valves were inserted and the syringes stored at −20° C. until testing for cytokine release. As the endpoints, either the cytokines IFN-gamma and IL-2, which are considered to be relevant in T-cell activation, were determined (by Simoa/Quanterix), or a spectrum of messenger substances was quantified in a multiplex assay (Luminex; see Table 4).

The data presented in Tables 1-4 below clearly demonstrate, on the basis of the reliably measurable tetanus-toxoid-induced or CEF-peptide-induced release of characteristic cytokines, the highly specific detection of functional antigen-specific activation of T cells using the kit.

TABLE 1

| | | | IFNg | | IL-2 | |
|---|---|---|---|---|---|---|
| | | CEF | Conc | | Conc | |
| | | [μg/ml] | [pg/mL] | SI | [pg/mL] | SI |
| CM 2 | Donor A | 0 | 365 | | 0.126 | |
| | | 1 | 1022 | 2.80 | 1.76 | 14.1 |
| | | 2 | 1515 | 4.15 | 3.07 | 24.5 |
| | | 4 | 1870 | 5.12 | 4.53 | 36.1 |
| | Donor B | | 8189 | | 0.272 | |
| | | 1 | 17986 | 2.20 | 6.85 | 24.1 |
| | | 2 | 23106 | 2.82 | 8.38 | 30.9 |
| | | 4 | 26953 | 3.29 | 8.25 | 30.4 |
| Stim. 2 | Donor A | | 38435 | | 0.109 | |
| | | 1 | 29038 | 0.755 | 1.28 | 11.8 |
| | | 2 | 28511 | 0.742 | 1.27 | 11.6 |
| | | 4 | 22252 | 0.579 | 1.46 | 13.4 |
| | Donor B | | 42685 | | 0.447 | |
| | | 1 | 38192 | 0.895 | 0.533 | 1.19 |
| | | 2 | 33448 | 0.784 | 0.672 | 1.50 |
| | | 4 | 31714 | 0.743 | 0.388 | 0.868 |

Cytokine release in whole blood culture supernatants of differently co-activated whole blood cultures under stimulation with CEF peptides (mixture of viral antigens that triggers an immunological memory response in approx. 90% of all blood donors).

The supernatants from 48 h cultures were analyzed using highly sensitive Simoa assays. CM=cytokine mix

TABLE 2

| | | IFNg | | IL-2 | |
|---|---|---|---|---|---|
| | | Conc | | Conc | |
| | | [pg/mL] | mean | [pg/mL] | mean |
| Donor A | Neg. Co. | <0.10 | <0.10 | <0.116 | <0.116 |
| | | <0.10 | | <0.116 | |
| | TeTo | 9.18 | 9.61 | 32.9 | 33.7 |
| | | 10.0 | | 34.4 | |
| | TeTo + | 7.62 | 7.64 | 26.2 | 26.4 |
| | Co-Stim. 1 | 7.66 | | 26.6 | |
| Donor B | Neg. Co. | <0.10 | <0.10 | <0.116 | <0.116 |
| | | <0.10 | | <0.116 | |
| | TeTo | 8.87 | 9.90 | 32.6 | 34.6 |
| | | 10.9 | | 36.6 | |
| | TeTo + | 8.13 | 8.58 | 27.0 | 25.9 |
| | Co-Stim. 1 | 9.02 | | 24.8 | |
| Donor C | Neg. Co. | 0.309 | 0.427 | <0.116 | <0.116 |
| | | 0.545 | | <0.116 | |
| | TeTo | 129 | 137 | 142 | 146 |
| | | 145 | | 149 | |
| | TeTo + | 77_{.}9 | 86.3 | 86.4 | 98.0 |
| | Co-Stim. 1 | 94.7 | | 110 | |

Cytokine release in the supernatants of tetanus-toxoid-activated whole blood cultures. The supernatants from 48 h cultures were analyzed using the Simoa assay.

TABLE 3

| | | | IFNg | | IL-2 | |
|---|---|---|---|---|---|---|
| | | | Conc | | Conc | |
| | | | [pg/mL] | SI | [pg/mL] | SI |
| Donor A | Medium | | 0.333 | | 0.023 | |
| | | TeTo | 1069 | 3208 | NQ | nb |
| | CM 3 | Medium | 309 | | 0.131 | |
| | | TeTo | 7105 | 23.0 | 212 | 1617 |
| | CM 2 | Medium | 347 | | 0.130 | |
| | | TeTo | 7833 | 22.6 | 153 | 1176 |
| | Stim. 2 | Medium | 33400 | | 0.130 | |
| | | TeTo | 148332 | 4.44 | 43.0 | 331 |
| Donor B | Medium | | 0.732 | | 0.039 | |
| | | TeTo | 11.3 | 15.4 | 12.1 | 314 |
| | CM 3 | Medium | 4669 | | 0.320 | |
| | | TeTo | 41010 | 8.78 | 6.70 | 20.9 |
| | CM 2 | Medium | 5993 | | 0.178 | |
| | | TeTo | 46177 | 7.71 | 4.93 | 27.7 |
| | Stim. 2 | Medium | 50768 | | 0.161 | |
| | | TeTo | 252303 | 4.97 | 5.50 | 34.2 |

Cytokine release in differently co-activated whole blood cultures under stimulation with tetanus toxoid. In this example, subjects recently vaccinated against tetanus were used as blood donors (hence the high IFNg levels). The supernatants from 48 h cultures were analyzed using the Simoa assay. For comparison of the various co-activators, the stimulation indices (SI) were additionally calculated, for which the ratio of the value for the unstimulated culture (“medium”) to that of the respective stimulated (TeTo) culture was calculated. nd=not determinable

TABLE 4

| | | Analy | GM-CSF | IFN-g | IL-10 | IL-18 | IL-2 | IL-3 | IL-4 | IL-5 |
|---|---|---|---|---|---|---|---|---|---|---|
| | | Units | pg/mL | pg/mL | pg/mL | pg/mL | pg/mL | ng/mL | pg/mL | pg/mL |
| | | LLOQ | 29.4 | 2.28 | 4.24 | 16.7 | 12.2 | 0.00186 | 18.9 | 7.28 |
| Donor A | Medium | Med. | <LOW> | <LOW> | <LOW> | 21.9 | 4.35 | <LOW> | <LOW> | <LOW> |
| | | TeTo | 144 | 204 | 8.24 | 37.5 | 580 | 0.0185 | 197 | 58.8 |
| | CM 3 | Med. | <LOW> | 74.2 | 35.5 | 29.8 | 8.74 | 0.00248 | 80.4 | <LOW> |
| | | TeTo | 425 | 1750 | 187 | 43.1 | 369 | 0.0295 | 151 | 25.4 |
| | CM 2 | Med. | <LOW> | 75.2 | 31.4 | 33.7 | 6.62 | 0.00339 | 77.4 | <LOW> |
| | | TeTo | 426 | 2020 | 203 | 41.3 | 310 | 0.0304 | 189 | 51.4 |
| | Co- | Med. | 157 | 6450 | 979 | 69.1 | 40.5 | 0.0199 | 79.4 | <LOW> |
| | Stim. 2 | TeTo | 939 | 28800 | 430 | 212 | 166 | 0.0399 | 181 | 8.57 |
| Donor B | Medium | Med. | <LOW> | 0.749 | 2.27 | 78.2 | <LOW> | <LOW> | <LOW> | <LOW> |
| | | TeTo | <LOW> | 3.55 | 8.47 | 83.7 | 27.8 | 0.00099 | 24.8 | <LOW> |
| | CM 3 | Med. | <LOW> | 1190 | 3.63 | 92.7 | <LOW> | 0.00309 | 41.4 | <LOW> |
| | | TeTo | <LOW> | 10600 | 6.85 | 85.5 | 32 | 0.0131 | 82.5 | <LOW> |
| | CM 2 | Med. | <LOW> | 1540 | 2.5 | 78.2 | 6.62 | 0.00445 | 47.6 | <LOW> |
| | | TeTo | <LOW> | 11400 | 6.85 | 90.9 | 21.4 | 0.0156 | 90.6 | <LOW> |
| | Co- | Med. | 14.6 | 14200 | 1580 | 125 | 53.1 | 0.0268 | 76.3 | <LOW> |
| | Stim. 2 | TeTo | 62.8 | 52900 | 2230 | 249 | 93.9 | 0.0418 | 148 | <LOW> |
| | | | IL-6 | IL-7 | IL-8 | MCP-1 | MIP-1 a | MIP-1 a | TNF-a | TNF-b |
| | | | pg/mL | pg/mL | pg/mL | pg/mL | pg/mL | pg/mL | pg/mL | pg/mL |
| | | | 3.27 | 10.7 | 3.88 | 26.9 | 17.3 | 28.3 | 10.4 | 5.76 |
| | Donor A | Medium | <LOW> | <LOW> | 91.1 | 111 | 7.11 | 133 | <LOW> | <LOW> |
| | | | 228 | 190 | >5700 | >25680 | 2450 | 6200 | 745 | 50.1 |
| | | CM 3 | 111 | 85 | >5700 | >25680 | 194 | >11550 | 6180 | 7.93 |
| | | | 13500 | 75.5 | >228000 | 139000 | 24200 | 260000 | 9840 | 71.6 |
| | | CM 2 | 109 | 92 | >5700 | >25680 | 207 | >11550 | 6070 | 6.44 |
| | | | 16200 | 79.6 | >228000 | 158000 | 26600 | 269000 | 9990 | 75.7 |
| | | Co- | 8480 | 69.9 | 6260 | 29000 | 15800 | 404000 | 9340 | 73.2 |
| | | Stim. 2 | 9820 | 71.3 | 7390 | 4960 | 25400 | 325000 | 22100 | 159 |
| | Donor B | Medium | 1.16 | <LOW> | 51.5 | 126 | 7.11 | 429 | <LOW> | <LOW> |
| | | | 21.2 | 31.8 | 833 | 5420 | 33 | 7050 | 28.3 | 2.15 |
| | | CM 3 | 14.4 | 37.3 | 1860 | 18100 | 186 | 29400 | 3840 | 6.44 |
| | | | 302 | 110 | 15400 | 103000 | 1380 | 111000 | 5280 | 41.8 |
| | | CM 2 | 14.1 | 36.4 | 1760 | 21300 | 219 | 33100 | 4760 | 7.93 |
| | | | 328 | 86.3 | 11900 | 90400 | 1490 | 107000 | 5990 | 50.9 |
| | | Co- | 11800 | 55.6 | 5810 | 38300 | 24600 | >462000 | 8210 | 69.9 |
| | | Stim. 2 | 22100 | 81 | 10000 | 17600 | 57300 | >462000 | 42800 | 135 |

Cytokine release in the supernatants of differently co-activated whole blood cultures under stimulation with tetanus toxoid (same test run as in Tab. 3). In this example, the supernatants from 48 h cultures were however determined by a multiplex cytokine analysis. CM=cytokine mix

Table 5 below shows the result of a whole blood analysis from two donors using a kit. The whole blood from the two donors was then analyzed to see whether the immune system specifically recognizes tetanus toxoid, the standard tetanus vaccine. The release of the messenger substances IFN-gamma (IFNg) and interleukin-2 (IL-2), which are regarded as typical for T-cell activation, was examined. The extent to which the signal-to-noise ratio can be improved through co-stimulating additives (such as cytokines) was at the same time examined.

TABLE 5

| | | | IFNg | IL-2 |
|---|---|---|---|---|
| Spender | Costim | Antigen | [pg/mL] | [pg/mL] |
| A | ohne | — | 0.333 | 0.023 |
| | | TetTox | 1069 | NQ |
| | A | — | 309 | 0.131 |
| | | TetTox | 7105 | 212 |
| | B | — | 347 | 0.130 |
| | | TetTox | 7833 | 153 |
| | C | — | 33400 | 0.130 |
| | | TetTox | 148332 | 43.0 |
| B | ohne | — | 0.732 | 0.039 |
| | | TetTox | 11.3 | 12.1 |
| | A | — | 4669 | 0.320 |
| | | TetTox | 41010 | 6.70 |
| | B | — | 5993 | 0.178 |
| | | TetTox | 46177 | 4.93 |
| | C | — | 50768 | 0.161 |
| | | TetTox | 252303 | 5.50 |

Result of whole blood analysis of two donors (TetTox=tetanus toxoid)

1-16. (canceled)

17. An in vitro method
for diagnostics including therapy-accompanying diagnostics, relating to a transplant, the prognostic assessment of prospects of success and/or predicting a clinical outcome of a transplant and/or determining suitability/effectiveness/dose of compounds, immunosuppressants, for a transplant, and/or therapy monitoring of a transplant,
and/or
for diagnostics including therapy-accompanying diagnostics, relating to an autoimmune disease, characterizing a stage of an autoimmune disease and/or prognostic assessment of the prospects of success and/or predicting a clinical outcome of a therapy of an autoimmune disease and/or determining the suitability and/or effectiveness and/or dose of compounds, immunosuppressants, for a therapy of an autoimmune disease, and/or therapy monitoring of an autoimmune disease,
and/or
for diagnostics including therapy-accompanying diagnostics, relating to a tumor disease, prognostic assessment of prospects of success and/or predicting a clinical outcome of a therapy of a tumor disease and/or determining suitability and/or effectiveness and/or dose of compounds for a therapy of a tumor disease and/or therapy monitoring of a tumor disease,
and/or
for diagnostics including therapy-accompanying diagnostics, relating to a vaccination, for prognostic assessment of the prospects of success and/or predicting a clinical outcome of a vaccination and/or therapy monitoring of a vaccination,
comprising the step of using a kit,
wherein the kit comprises:
a blood collection container, a syringe, or a blood collection set or blood collection system including a blood collection container or a syringe barrel,
a nutrient medium for blood, and
at least one activator that activates blood cells or immune cells, and/or at least one inhibitor that inhibits blood cells or immune cells, and/or at least one modulator that modulates blood cells or immune cells.

18. The in vitro method as claimed in claim 17, wherein the at least one activator that activates blood cells or immune cells, is selected from the group consisting of cells from a transplant donor, antigen preparations of cells from a transplant donor, antigens, superantigens, autoantigens, tumor antigens, tumor vaccines, mitogens, ligands of blood cell surface receptors that have an activating or inhibitory effect on blood cells, ligands of intracellular receptors of blood cells that have an activating or inhibitory effect on blood cells, ligands of ion channels that have an activating or inhibitory effect on blood cells and are accessible via surface receptors or intracellular receptors of blood cells, ligands of enzymes that have an activating or inhibitory effect on blood cells and are accessible via surface receptors or intracellular receptors of blood cells, antibodies to blood cell surface receptors that have an activating or inhibitory effect on blood cells, antibodies to intracellular receptors of blood cells that have an activating or inhibitory effect on blood cells, antibodies to ion channels that have an activating or inhibitory effect on blood cells and are accessible via surface receptors or intracellular receptors of blood cells, antibodies to enzymes that have an activating or inhibitory effect on blood cells and are accessible via surface receptors or intracellular receptors of blood cells, adjuvants, and combinations or mixtures, of at least two of the activators.

19. The in vitro method as claimed in claim 17, wherein
the cells of the transplant donor are organ cells selected from the group consisting of blood cells, skin cells, kidney cells, liver cells, heart cells, lung cells, pancreas cells, small intestine cells, bone cells, bone marrow cells, and combinations or mixtures, of at least two of the cells from a transplant donor,
and/or
the antigen preparations of cells from a transplant donor are selected from the group consisting of intact cells, cell membrane fragments, microsomes, cell- and/or cell-fragment-free filtrates, ultrafiltrates, extracts, and combinations or mixtures, of at least two of the antigen preparations of cells from a transplant donor,
and/or
the superantigens are selected from the group consisting of staphylococcal enterotoxins such as staphylococcal enterotoxin A and/or staphylococcal enterotoxin B and/or staphylococcal enterotoxin C, toxic shock syndrome toxin, Streptococcus pyogenes exotoxin, and combinations or mixtures, of at least two of the superantigens,
and/or
the autoantigens are selected from the group consisting of myelin basic protein (MBP), outer surface protein (OspA), leukocyte function-associated antigen 1α (LFA-1α), acetylcholinesterase receptor antigen (AChR), and combinations or mixtures, of at least two of the autoantigens,
and/or
the tumor antigens are selected from the group consisting of MAGE, BAGE, GAGE, CTAG, NY-ESO, MUC-1, CDK4, beta-catenin, and combinations or mixtures, of at least two of the tumor antigens,
and/or
the tumor vaccines are combinations of a tumor antigen and an adjuvant, or a combination of PAP and GMCSF or a combination of MUC-1 and monophosphoryl lipid A,
and/or
the mitogens are selected from the group consisting of phytohemagglutinin, pokeweed mitogen, concanavalin A, and combinations or mixtures, of at least two of the mitogens,
and/or
the ligands of blood cell surface receptors that have an activating or inhibitory effect on blood cells are selected from the group consisting of ligands of CD2, ligands of CD3, ligands of CD11, ligands of CD16, ligands of CD18, ligands of CD32, ligands of CD64, and combinations or mixtures, of at least two of the ligands,
and/or
the ligands of intracellular receptors of blood cells that have an activating or inhibitory effect on blood cells are selected from the group consisting of TLR ligands, NOD ligands, RIG ligands, PPAR-γ ligands, and combinations or mixtures, of at least two of the ligands,
and/or
the antibodies to blood cell surface receptors that have an activating or inhibitory effect on blood cells are selected from the group consisting of antibodies to CD2, antibodies to CD3, antibodies to CD11, antibodies to CD16, antibodies to CD18, antibodies to CD32, antibodies to CD64, and combinations or mixtures, of at least two of the antibodies,
and/or
the antibodies to intracellular receptors of blood cells that have an activating or inhibitory effect on blood cells are selected from the group consisting of antibodies to TLR9, antibodies to NOD, antibodies to RIG, antibodies to PPAR-γ, and combinations or mixtures, of at least two of the antibodies.

20. The in vitro method as claimed in claim 17, wherein the at least one inhibitor that inhibits blood cells or immune cells, is selected from the group consisting of anti-inflammatory compounds/drugs, immunosuppressants, and combinations or mixtures, of at least two of the inhibitors, when the kit is used in vitro for diagnostics including therapy-accompanying diagnostics, relating to a transplant and/or an autoimmune disease, for the prognostic assessment of the prospects of success and/or predicting a clinical outcome of a transplant and/or a therapy of an autoimmune disease and/or determining the suitability and/or effectiveness and/or dose of compounds, immunosuppressants, for a transplant and/or of an autoimmune disease, and/or therapy monitoring of a transplant and/or an autoimmune disease.

21. The in vitro method as claimed in claim 20, wherein
the anti-inflammatory compounds/drugs are selected from the group consisting of cortisones, inhibitors of the synthesis of arachidonic acid metabolites, kinase inhibitors, inhibitors of cyclooxygenases, inhibitors of lipoxygenases, receptor blockers, siRNA, ion-channel blockers, and combinations or mixtures, of at least two of the anti-inflammatory compounds/drugs,
and/or
the immunosuppressants are selected from the group consisting of glucocorticoids, mTOR inhibitors, cytostatics, antimetabolites, monoclonal antibodies, anti-T-lymphocyte globulin, mycophenolates, and combinations or mixtures, of at least two of the immunosuppressants.

22. The in vitro method as claimed in claim 17, wherein the at least one modulator that modulates blood cells or immune cells, is selected from the group consisting of methotrexate, azathioprine, 6-mercaptopurine, leflunomide, sulfasalazine, chloroquine, checkpoint inhibitors, calcineurin inhibitors, DMARDs (disease-modifying antirheumatic drugs), TLR agonists, and combinations or mixtures, of at least two of the modulators.

23. The in vitro method as claimed in claim 17, wherein the kit also includes at least one co-activator selected from the group consisting of ligands of CD (cluster of differentiation) surface molecules, antibodies to CD (cluster of differentiation) surface molecules, TLR ligands, NOD ligands, cytokines and combinations, more particularly mixtures, of at least two of the co-activators, and/or also at least one co-inhibitor selected from the group consisting of ligands of CD (cluster of differentiation) molecules, antibodies to CD (cluster of differentiation) molecules, and combinations or mixtures, of at least two of the co-inhibitors, and/or also at least one co-modulator selected from the group consisting of inhibitors of enzymes of tryptophan degradation such as TDO and/or IDO, modulating metabolites, ligands, antibodies of receptors, inhibitors or allosteric regulators of enzymes, enzymes from signal transduction cascades, inhibitors or activators of ion channels, and combinations or mixtures, of at least two of the co-modulators.

24. The in vitro method as claimed in claim 17, wherein the transplant is an organ transplant, more particularly a kidney transplant, liver transplant, heart transplant, lung transplant, pancreas transplant, small intestine transplant, bone marrow transplant, bone transplant or skin transplant.

25. The in vitro method as claimed in claim 17, wherein
the autoimmune disease is selected from the group consisting of alopecia areata, autoimmune enteropathy, autoimmune hepatitis, APECED, bullous pemphigoid, chronic type A gastritis, eosinophilic granulomatosis with polyangiitis, CIDP, chronic active hepatitis, ulcerative colitis, dermatomyositis, type 1 diabetes mellitus, dermatitis herpetiformis (Duhring's disease), endocrine orbitopathy, epidermolysis bullosa acquisita, glomerulonephritis, Goodpasture syndrome, granulomatosis with polyangiitis, Guillain-Barré syndrome, Hashimoto's thyroiditis, idiopathic thrombocytopenic purpura, lichen sclerosus, lichen mucosae, linear IgA dermatosis, lupus erythematosus, microscopic polyangiitis, Graves' disease, Behcet's disease, Crohn's disease, ankylosing spondylitis, multiple sclerosis, myasthenia gravis, narcolepsy, PANDAS, pemphigus foliaceus, pemphigus seborrhoicus, pemphigus vulgaris, polychondritis, polymyalgia rheumatica, polymyositis, psoriasis, primary biliary cirrhosis, primary chronic polyarthritis, rheumatic fever, rheumatoid arthritis, giant cell arteritis, SAPHO syndrome, sarcoidosis (Boeck's disease), Sjögren's syndrome, scleroderma, stiff-person syndrome, sympathetic ophthalmia, systemic lupus erythematosus, Henoch-Schönlein purpura, Wegener's granulomatosis, vitiligo, and celiac disease,
and/or
the tumor disease is selected from the group consisting of carcinoma, melanoma, blastoma, lymphoma, leukemia, and sarcoma,
and/or
the vaccination is selected from the group consisting of antiviral vaccination, antibacterial vaccination, antifungal vaccination, antiparasitic vaccination, anti-autoimmunogical vaccination and antitumor vaccination.

26. The in vitro method as claimed in claim 17, wherein
the at least one activator that activates blood cells or immune cells, and/or the at least one inhibitor that inhibits blood cells or immune cells, and/or the at least one modulator that modulates blood cells or immune cells, are/is present in liquid or dissolved form,
and/or
the at least one activator that activates blood cells or immune cells, and/or the at least one inhibitor that inhibits blood cells or immune cells, and/or the at least one modulator that modulates blood cells or immune cells, are/is present in the blood collection container,
and/or
the nutrient medium is present in the blood collection container.

27. The in vitro method as claimed in claim 17, wherein
the blood collection container or the syringe, has a hollow cylinder in which a plunger is arranged such that it can be displaced with a plunger rod to create a seal, and a closure cap for closing the hollow cylinder, and the closure cap has an attachment socket for a cannula,
and/or
the blood collection set/blood collection system comprises, in addition to the syringe, a cannula or a butterfly cannula, and/or blood collection tubing,
and/or
the kit also comprises a component selected from the group consisting of anticoagulant, thermostatically controllable device such as a thermoblock, a number of cooling blocks, a refrigerator, a freezer, a container with dry ice, a nitrogen tank, and combinations of at least two of the components.

28. The in vitro method as claimed in claim 17, wherein the kit also comprises a separating component associated with the blood collection container for the separation of a supernatant and sediment formed in the blood collection container.

29. The in vitro method as claimed in claim 28, wherein the separating component comprises a valve or a valve plunger that has a valve and a pusher, wherein the valve is open when the valve or the valve plunger is connected to the pusher, and the valve is closed when the valve or the valve plunger are separated from one another.

30. The in vitro method
for diagnostics including therapy-accompanying diagnostics, relating to a transplant, prognostic assessment of prospects of success and/or predicting a clinical outcome of a transplant and/or determining suitability and/or effectiveness and/or dose of compounds, immunosuppressants, for a transplant, and/or therapy monitoring of a transplant,
and/or
for diagnostics including therapy-accompanying diagnostics, relating to an autoimmune disease characterizing a stage of an autoimmune disease and/or prognostic assessment of prospects of success and/or for predicting a clinical outcome of a therapy of an autoimmune disease and/or determining suitability and/or effectiveness and/or dose of compounds, immunosuppressants, for a therapy of an autoimmune disease, and/or therapy monitoring of an autoimmune disease,
and/or
for diagnostics including therapy-accompanying diagnostics, relating to a tumor disease for prognostic assessment of prospects of success and/or predicting a clinical outcome of a therapy of a tumor disease and/or determining suitability and/or effectiveness and/or dose of compounds for a therapy of a tumor disease and/or therapy monitoring of a tumor disease,
and/or
for diagnostics including therapy-accompanying diagnostics, relating to a vaccination for prognostic assessment of prospects of success and/or predicting a clinical outcome of a vaccination and/or therapy monitoring of a vaccination,
wherein the method comprises:
a) preparing a mixture comprising whole blood, a nutrient medium for whole blood, and at least one activator that activates blood cells or immune cells, in addition to any activators present in the whole blood, and/or at least one inhibitor that inhibits blood cells or immune cells, in addition to any inhibitors present in the whole blood, and/or at least one modulator that modulates blood cells or immune cells, in addition to any modulators present in the whole blood,
b) incubating the prepared mixture,
c) separating a messenger-substance-containing supernatant formed through incubation of the prepared mixture from a cell-containing sediment formed through incubation of the prepared mixture, or a messenger-substance-containing supernatant formed through centrifugation of the incubated prepared mixture from a cell-containing sediment formed through centrifugation of the incubated prepared mixture, and
d) detecting and/or determining messenger substances present in the supernatant and/or determining cell surface molecules and/or intracellular activation markers of blood cells or immune cells, present in the sediment.

31. An in vitro method for
diagnostics including therapy-accompanying diagnostics, relating to a transplant in prognostic assessment of prospects of success and/or in predicting a clinical outcome of a transplant and/or in determining the suitability and/or effectiveness and/or dose of compounds, immunosuppressants, for a transplant, and/or in therapy monitoring of a transplant,
and/or
diagnostics including therapy-accompanying diagnostics, relating to an autoimmune disease, characterizing a stage of an autoimmune disease and/or in prognostic assessment of prospects of success and/or in predicting a clinical outcome of a therapy of an autoimmune disease and/or in determining suitability and/or effectiveness and/or dose of compounds or immunosuppressants, for a therapy of an autoimmune disease, and/or in therapy monitoring of an autoimmune disease,
and/or
diagnostics including therapy-accompanying diagnostics, relating to a tumor disease, in prognostic assessment of prospects of success and/or in predicting a clinical outcome of a therapy of a tumor disease and/or in determining suitability and/or effectiveness and/or dose of compounds for a therapy of a tumor disease, and/or in therapy monitoring of a tumor disease,
and/or
diagnostics including therapy-accompanying diagnostics, relating to a vaccination in prognostic assessment of prospects of success and/or in predicting a clinical outcome of a vaccination and/or in therapy monitoring of a vaccination,
comprising using a messenger-substance-containing supernatant or cell-containing sediment, wherein the messenger-substance-containing supernatant or the cell-containing sediment is produced or producible by a method comprising:
a) preparing a mixture comprising whole blood, a nutrient medium for whole blood, and at least one activator that activates blood cells or immune cells, in addition to any activators present in the whole blood, and/or at least one inhibitor that inhibits blood cells or immune cells, in addition to any inhibitors present in the whole blood, and/or at least one modulator that modulates blood cells or immune cells, in addition to any modulators present in the whole blood,
b) incubating the prepared mixture, and
c) separating a messenger-substance-containing supernatant formed through incubation of the prepared mixture from a cell-containing sediment formed through incubation of the prepared mixture, or a messenger-substance-containing supernatant formed through centrifugation of the incubated prepared mixture from a cell-containing sediment formed through centrifugation of the incubated prepared mixture.

32. An in vitro method for
diagnostics including therapy-accompanying diagnostics, relating to a transplant in prognostic assessment of prospects of success and/or in predicting a clinical outcome of a transplant and/or in determining suitability and/or effectiveness and/or dose of compounds or immunosuppressants, for a transplant, and/or in therapy monitoring of a transplant,
and/or
diagnostics including therapy-accompanying diagnostics, relating to an autoimmune disease characterizing the stage of an autoimmune disease and/or in prognostic assessment of prospects of success and/or in predicting a clinical outcome of a therapy of an autoimmune disease and/or in determining suitability and/or effectiveness and/or dose of compounds or immunosuppressants, for a therapy of an autoimmune disease, and/or in therapy monitoring of an autoimmune disease,
and/or
diagnostics including therapy-accompanying diagnostics, relating to a tumor disease in prognostic assessment of prospects of success and/or in predicting a clinical outcome of a therapy of a tumor disease and/or in determining suitability and/or effectiveness and/or dose of compounds for a therapy of a tumor disease, and/or in therapy monitoring of a tumor disease,
and/or
diagnostics including therapy-accompanying diagnostics, relating to a vaccination in prognostic assessment of prospects of success and/or in predicting a clinical outcome of a vaccination and/or in therapy monitoring of a vaccination,
comprising using a combination or a mixture, comprising whole blood, a nutrient medium for whole blood, and at least one activator that activates blood cells or immune cells, in addition to any activators present in the whole blood, and/or at least one inhibitor that inhibits blood cells or immune cells, in addition to any inhibitors present in the whole blood, and/or at least one modulator that modulates blood cells or immune cells, in addition to any modulators present in the whole blood.
